(19) United States
(12) Patent Application Publication
(10) Pub. No.: US 2024/0118281 A1
(43) Pub. Date: Apr. 11, 2024

(54) ANTICANCER COMPOUNDS AND USES THEREOF

(71) Applicant: University of Florida Research Foundation, Incorporated, Gainesville, FL (US)

(72) Inventors: Brian Keith Law, Gainesville, FL (US); Ronald K. Castellano, Gainesville, FL (US)

(73) Assignee: University of Florida Research Foundation, Incorporated, Gainesville, FL (US)

(21) Appl. No.: 18/271,584

(22) PCT Filed: Jan. 11, 2022
(86) PCT No.: PCT/US2022/011961

§ 371 Date: Jul. 10, 2023

Related U.S. Application Data
(60) Provisional application No. 63/135,979, filed on Jan. 11, 2021.

(51) Int. Cl. G01N 33/574; A61P 35/00; C07D 339/08; C07D 495/04; C07D 519/00

(52) U.S. Cl. CPC G01N 33/57415; A61P 35/00; C07D 339/08; C07D 495/04; C07D 519/00

(57) ABSTRACT

Provided are compositions and methods of treating cell proliferative disorders, especially cancer. Also provided are methods for assessing and monitoring cell proliferative processes and processes for amelioration of cell proliferative disorders, especially cancer.

### RELATED APPLICATIONS

This application claims priority under 35 U.S.C. § 119(e) to U.S. Provisional Application, Ser. No. 63/135,979, filed on Jan. 11, 2021, the content of which is incorporated herein by reference in its entirety.

### STATEMENT OF GOVERNMENT SUPPORT

This invention was made with government support under Grant No. CA252400 awarded by the National Institutes of Health. The government has certain rights in the invention.

### BACKGROUND

In spite of significant improvements in early detection, and multiple new molecularly targeted therapeutics, breast cancer remains the second largest cancer killer of women in America after lung cancer and is the most frequently diagnosed cancer in American women. Much of the lethality of breast cancer derives from an inability to successfully treat patients with metastatic and/or drug-resistant malignancies. New drugs are needed that bypass resistance to currently used regimens by targeting cancer cell-specific vulnerabilities. A class of agents termed Disulfide bond Disrupting Agents (DDAs) was previously identified and shown to kill breast cancer cells in association with downregulation of the HER-family proteins EGFR, HER2, and HER3, and decreased activating phosphorylation of the Akt kinase. Subsequent work showed that DDAs induce endoplasmic reticulum stress (ERS) and alter the pattern of disulfide bonding of EGFR and the TRAIL receptors, Death Receptor 4 (DR4) and Death Receptor 5 (DR5). DDA-induced cell death is mediated by activation of the Caspase 8-Caspase 3 extrinsic apoptotic pathway.

Little is known regarding how the disulfide bonding of HER-family and Death Receptor-family proteins are facilitated within the endoplasmic reticulum, and how the disulfide bonding patterns of these proteins control their stability, intracellular localization, and downstream signaling. TNF-Related Apoptosis Inducing Ligand (TRAIL) has been considered a promising cancer therapeutic agent due to its selectivity for killing cancer cells, while leaving normal tissues unharmed. However, TRAIL analogs and DR4/5 agonistic antibodies have yet to be clinically approved for anticancer therapy. This is due in part to pharmacokinetic issues related to these protein drugs themselves, and in part to the ability of cancer cells to acquire resistance by downregulating DR4 and DR5. Since DDAs upregulate DR5 and activate DR4 and DR5 in a ligand-independent manner, DDAs may overcome the resistance mechanisms that suppress the efficacy of other activators of the TRAIL/Death Receptor pathway. The extracellular domain of DR5 prevents oligomerization and downstream activation of Caspase 8 in the absence of ligand engagement, and excision of the DR5 extracellular domain permits full activation of DR5 in a ligand-independent manner. It was previously proposed that DDAs activate DR5 in a ligand-independent manner by permitting inter-molecular disulfide bond formation at the expense of intramolecular disulfide bonding, resulting in DR5 oligomerization and activation. However, the precise mechanisms through which this happens are unknown. Similarly, DDA treatment causes disulfide bond-dependent EGFR oligomerization followed by degradation, but how DDAs perturb EGFR disulfide bonding is unknown. The Protein Disulfide Isomerase (PDI) family member AGR2 is essential for EGFR folding and surface localization, and likewise facilitates expression of Mucins.

The PDI family contains 21 members. Although significant attention has focused on the roles of canonical PDIs in mediating disulfide bond formation via their active site thioredoxin-like CXXC motifs, and canonical PDIs are considered promising targets for anticancer therapeutics, the other PDI family members are less well studied. The PDI proteins AGR2 and AGR3 contain a non-canonical CXXS active site motif. Based on biochemical PDI mutagenesis studies it is predicted that AGR2/3 will exhibit disulfide isomerase activity, but not oxidase or reductase activity. Consistent with this, AGR2 forms stable mixed disulfide bonds with its client proteins. Another PDI family member, ERp44, also contains an active site CXXS motif, and has multiple functions within the endoplasmic reticulum (ER). ERp44 plays an important role in the disulfide-mediated oligomerization of Adiponectin and Immunoglobulin M. Additionally, ERp44 anchors client/partner proteins such as ERO1α, Prx4, FGE/SUMF1, and ERAP1 that do not contain their own C-terminal -KDEL ER retention sequences to prevent their secretion.

### SUMMARY

HER-family proteins are the only sub-family of receptor tyrosine kinases that contain two cysteine-rich repeats in their extracellular domain. DR4 and DR5 also harbor numerous disulfide bonds in their extracellular domains. Based on these observations, HER-family proteins and DR4/5 may be particularly sensitive to agents that impede native disulfide bond formation. Despite the thiol-reactivity of DDAs, the mechanisms by which they cause downregulation of EGFR, HER2, HER3 and activation of DR4/5 are unknown. Disclosed herein are biotinylated DDA derivatives that demonstrate that DDAs covalently modify AGR2, PDIA1, and ERp44 in vitro and in intact cells, and that DDA treatment of cells disrupts PDIA1 and ERp44 disulfide bonds with their client proteins. Together, the results indicate that DDAs activate DR4/5 and inactivate EGFR, HER2, and HER3 by inhibiting the activity of the PDI family members AGR2, PDIA1, and ERp44, which mediate native disulfide bonding of these transmembrane receptors. DDAs are thus the first agents that function in part by modifying the active site Cys residues of AGR2 and ERp44 and demonstrate that ERp44 is a new molecular target for anticancer therapeutics.

The present disclosure stems from the recognition that there currently exist no testing procedures to predict tumor sensitivity to PDI inhibitors by analyzing tumor biopsies, or tests to monitor the biochemical efficacy of PDI inhibitors in vivo. Thus, the present disclosure demonstrates that tumors sensitive to PDIs are those in which the PDI proteins ERp44 and PDIA1 are fully engaged in disulfide bonds with client proteins, leaving little or no monomeric ERp44 or PDIA1. The ratio of oligomeric to monomeric ERp44 and PDIA1 is predictive of tumor sensitivity to ERp44 or PDIA1 inhibitors, and this this can be examined by non-reducing immunoblot of tumor biopsies.

Furthermore, additional disulfide bond-rich proteins, including EGFR, HER2, HER3, DR4, and DR5, when combined with ERp44 and PDIA1 will generate a highly sensitive readout of the state of cellular redox and protein disulfide bonding status. This status, in turn, provides a highly accurate indicator of tumor sensitivity to inhibitors of ERp44, PDIA1, and other PDI-family proteins.

In one aspect, provided are methods of determining the ratio of oligomeric forms of one of ERp44, PDIA1, or AGR2 relative to the monomeric form of that same protein in a biological sample of a subject, the method comprising: (a) measuring the amount of oligomeric forms of one of ERp44, PDIA1, or AGR2 in the subject sample; (b) measuring the amount of monomeric form of the same protein measured in step (a) in the subject sample; and (c) comparing the measurement of (a) and the measurement of (b) to determine the ratio of oligomeric forms to monomeric form.

In another aspect, provided are methods of lowering the ratio of oligomeric forms of one of ERp44, PDIA1, or AGR2 relative to the monomeric form of that same protein in a biological sample of a subject, the method comprising administering a compound to the subject, wherein the subject is determined to have a high ratio of oligomeric forms of one of ERp44, PDIA1, or AGR2 relative to the monomeric form of that same protein.

In another aspect, provided are methods of treating a patient comprising administering a cancer therapy to the patient, wherein the patient is determined to have a high ratio of oligomeric forms of one of ERp44, PDIA1, or AGR2 relative to the monomeric form of that same protein.

In another aspect, provided are test panels comprising an agent to assess the presence of ERp44 and/or an agent to assess the presence of PDIA1 in a subject sample.

In another aspect, provided are methods of testing for the presence of ERp44 and/or PDIA1 in a subject sample comprising contacting the test panel with a subject sample.

In another aspect, provided are compounds of Formula (III):

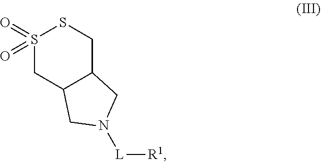

or a salt, solvate, hydrate or prodrug thereof, wherein:
L is a bond, substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, or —OC(═O)N(R^{A})—, or a combination thereof;
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or two R^{A} groups are joined to form a substituted or unsubstituted heterocyclic ring; and
R¹ is hydrogen, a nitrogen protecting group, or a label.

In certain embodiments, examples of the compound of Formula (III), include, but are not limited to compounds of the formula:

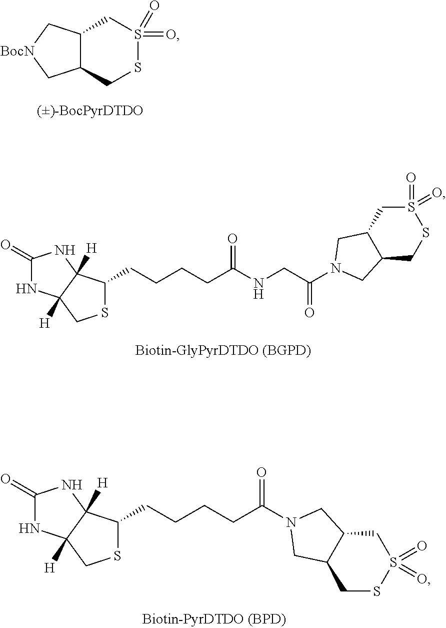 [SMILES: CC(C)(C)OC(=O)N1C[C@@H]2CS(=O)(=O)SC[C@H]2C1 ; O=C(CCCC[C@@H]1SC[C@@H]2NC(=O)N[C@@H]21)NCC(=O)N1C[C@@H]2CS(=O)(=O)SC[C@H]2C1 ; O=C1N[C@H]2[C@H](CS[C@H]2CCCCC(=O)N2C[C@@H]3CS(=O)(=O)SC[C@H]3C2)N1]

or a salt, solvate, hydrate or prodrug thereof.

Other aspects and embodiments of the invention are disclosed infra.

### BRIEF DESCRIPTION OF THE DRAWINGS

The present disclosure is further described below with reference to the following non-limiting examples and with reference to the following figures, in which:

FIG. 1 shows that DDAs induce rapid tumor regression and exhibit oral activity against metastatic disease. FIG. 1A. Representative first and second generation DDAs (RBF3, DTDO, and tcyDTDO) and illustration of interconversion between the linear and cyclic forms of the DDA pharmacophore. FIG. 1B. Mice bearing HCI-001/LVM2 breast tumors and associated liver metastases were treated once daily for four days with 20 mg/kg tcyDTDO. Livers were collected and stained with hematoxylin and eosin (H&E). FIG. 1C. Structures of new tcyDTDO derivatives dMtcyDTDO and dFtcyDTDO. FIG. 1D. MDA-MB-468 cells were treated for 24 h with the indicated concentrations of DDAs and cell viability (MTT) assays were performed. FIG. 1E. MDA-MB-468 cells were treated for 24 h as indicated and subjected to immunoblot analysis after non-reducing SDS-PAGE. O and M represent disulfide bonded oligomeric and monomeric protein forms, respectively. FIG. 1F. Levels and degree of oligomerization of DR4 (left panel) and DR5 (right panel) were determined by densitometry analysis of bands across multiple experiments and subjected to statistical analysis. Values plotted represent averages and error bars represent S.E. P-values for pairwise comparisons were determined using Student's t-test. For each concentration (0, 1.25, 2.5, 5 uM) of dMtcyDTO in the two bar plots, the bar on the left for each concentration represents monomer, the bar in the middle for each concentration represents oligomer and the bar on the right for each concentration represents % oligomerization. The top three P-values are for % oligomerization. The middle three P-values are for oligomer. The bottom three P-values are for monomer. FIG. 1G. Mice bearing BT474 cell xenograft tumors were treated once daily for five days with vehicle, 20 mg/kg dFtcyDTDO, or 20 mg/kg dMtcyDTDO. Two hours after the final treatment, mammary fat pads/tumors were excised and tumor sections were stained with H&E. T denotes tumor tissue, FP indicates mammary fat pads, and CN represents coagulation necrosis.

FIG. 2 shows that AGR2 and AGR3 are DDA target proteins. FIG. 2A. Abbreviated synthetic approach and chemical structures of the biotinylated DDA analogs Biotin-PyrDTDO and Biotin-GlyPyrDTDO. FIG. 2B. Purified, recombinant AGR2 was reacted with Biotin-GlyPyrDTDO for 1 h at 37° C., resolved by non-reducing SDS-PAGE, transferred to membranes, and probed with Streptavidin-Alkaline Phosphatase, and by anti-AGR2 immunoblot. FIG. 2C. Vector control or AGR2 knockdown MDA-MB-468 cells were treated for 24 h as indicated and analyzed by non-reducing (left panels) or reducing (right panels) immunoblot (CC8, Cleaved Caspase 8). Red arrow indicates oligomerized forms of DR5 that are present at higher levels in the AGR2 knockdown cells. O and M represent disulfide bonded oligomeric and monomeric protein forms, respectively. FIG. 2D. Biotin-GlyPyrDTDO binding assays performed as in FIG. 2B, employing the C81S AGR2 mutant and AGR3 in addition to AGR2. In the indicated samples, the proteins were pre-incubated with 600 μM dMtcyDTDO to determine if this prevented subsequent Biotin-GlyPyrDTDO binding. FIG. 2E. AGR2 binding assay performed as in FIG. 2B to examine the relative abilities of pre-incubation of AGR2 with dMtcyDTDO or tcyDTDO to block subsequent binding of Biotin-GlyPyrDTDO. FIG. 2F. The indicated MDA-MB-468 stable cell lines were treated for 24 h with 5 μM dMtcyDTDO and subjected to analysis by non-reducing SDS-PAGE/immunoblot. O, D, and M represent the oligomeric, dimeric, and monomeric forms of AGR2 detected. P43/p41 and p18 denote different Caspase 8 cleavage products.

FIG. 3 shows that ERp44 and PDIA1 are additional DDA targets. FIG. 3A. Breast cancer cell lines were treated for 16 h with vehicle, 100 μM Biotin-PyrDTDO, or 100 μM Biotin-PyrDTDO+10 μM Q-VD-OPH for 16 h. Biotinylated proteins were isolated using Streptavidin-coated beads and the crude extracts and Streptavidin pulldowns were analyzed by blotting with Streptavidin-Alkaline Phosphatase. Red arrows indicate bands, designated DDAT1 and DDAT2, present exclusively after Biotin-PyrDTDO treatment. FIG. 3B. Biotin-PyrDTDO/Streptavidin pulldowns were performed in BT474 and MDA-MB-468 cells. Purified material was analyzed by Streptavidin-Alkaline Phosphatase detection and silver stain. FIG. 3C, left panel. The indicated breast cancer lines were treated with vehicle or 100 μM Biotin-PyrDTDO for 16 h and extracts were subjected to Streptavidin-agarose pulldowns, or anti-AGR2 or anti-ERp44 immunoprecipitations. The purified material was analyzed by immunoblot with the indicated antibodies. FIG. 3C, right panel. BT474 and MDA-MB-468 breast cancer lines were treated with vehicle or 100 μM Biotin-PyrDTDO for 16 h and extracts were subjected to Streptavidin-agarose pulldowns. The affinity-purified samples were analyzed by immunoblot. FIG. 3D. Binding assays employing recombinant, purified ERp44 and PDIA1. Proteins were labeled with Biotin-PyrDTDO after pretreatment with vehicle or dMtcyDTDO. Reactions were analyzed by non-reducing SDS-PAGE followed by detection with Streptavidin-Alkaline Phosphatase. FIG. 3E. MDA-MB-468 cells were treated or 24 h with vehicle (control) or 5 μM dMtcyDTDO. Cell extracts were resolved by non-reducing SDS-PAGE and analyzed by immunoblot with the indicated antibodies. O and M represent disulfide bonded oligomeric and monomeric protein forms, respectively (CC8, Cleaved Caspase 8). Red arrows denote bands stained with ERp44 or PDIA1 antibodies that change in abundance with dMtcyDTDO treatment.

FIG. 4 shows that DDAs block ER retention of ERAP1 by ERp44. FIG. 4A. Model for the role of ERp44 in retrotranslocation of client proteins from the Golgi to the ER and retention of partner proteins within the ER. FIG. 4B. Immunoblot analysis of the levels of ERAP1 and MHC I in MDA-MB-468 cells after treatment with dMtcyDTDO for 24 h. FIG. 4C. Quantitative analysis of cellular ERAP1 levels in control and dMtyDTDO treated MDA-MB-468 cells across four experiments. FIG. 4D. Quantitative analysis of cellular ERp44 binding to client proteins in control and dMtyDTDO treated MDA-MB-468 cells across four experiments. FIG. 4E. The indicated stable MDA-MB-468 cell lines were treated for 24 h with dFtcyDTDO and subjected to non-reducing SDS-PAGE followed by immunoblot. O and M represent oligomeric and monomeric protein isoforms. FIG. 4F. MDA-MB-468 (left panels) or A431 cells (right panels) were treated for 24 h with 5 μM dMtcyDTDO and cell extracts and conditioned culture media were subjected to immunoblot analysis as indicated. ERAP1 levels in the cell extracts and culture medium were subjected to statistical analysis (lower panels, N=3).

FIG. 5 shows evidence that disulfide bonding of DR4 and DR5 are regulated by ERp44. FIG. 5A. Immunoblot analysis of the expression levels of EGFR, ERO1-α, AGR2, AGR3, PDIA1 and ERp44 across a panel of breast cancer cell lines. Actin serves as a loading control. FIG. 5B. MDA-MB-468 cells were treated for 24 h with 10 μM dMtcyDTDO or vehicle and cell extracts were immunoprecipitated with ERp44 antibody and analyzed by immunoblot. FIG. 5C. BT474 cells stably expressing ERp44 or transduced with the vector control were treated for 24 h with vehicle, 2.5 μM, or 5 μM dMtcyDTDO. Cell extracts were resolved by SDS-PAGE under non-reducing (left panel) or reducing (right panel) conditions and analyzed by immunoblot. O and M are disulfide bonded oligomeric and monomeric protein forms, respectively. FIG. 5D, upper panel. Vector control or ERp44 knockdown MDA-MB-468 cell lines were treated for 24 h with vehicle, 2.5 μM, or 5 μM dMtcyDTDO. Cell extracts were resolved by non-reducing SDS-PAGE and analyzed by immunoblot (CC8, Cleaved Caspase 8). O and M are disulfide bonded oligomeric and monomeric protein bands, respectively. FIG. 5D, lower panel. Expanded portions of the blots from the upper panel showing bands at the stacking gel/resolving gel interface (SGI). Asterisks denote bands at the SGI that are more prominent in the shERp44 knockdown cells.

FIG. 6 shows mutation of ERp44 thioredoxin-like repeat residues alters DDA responses. FIG. 6A. Non-reducing immunoblot analysis of vector control MDA-MB-468 cells, or cells overexpressing wild type ERp44, ERp44[C58S], or ERp44[S61C]. O and M are disulfide bonded oligomeric and monomeric protein bands, respectively. M (FL) and M (Cl.) represent the monomeric full-length and cleaved forms of CDCP1, respectively. FIG. 6B. Non-reducing immunoblot analysis of the indicated cell lines treated with vehicle or 7.5 μM dFtcyDTDO for 24 h. O and M are disulfide bonded oligomeric and monomeric protein bands, respectively. FIG. 6C. Extracts from vector control or EGFR overexpressing T47D cells were analyzed by non-reducing SDS-PAGE followed by immunoblot. O and M are disulfide bonded oligomeric and monomeric protein bands, respectively. FIG. 6D. MDA-MB-468 cells were treated for 24 h with vehicle or 2.5 μM dMtcyDTDO. The cells were then subjected to cell surface protein labeling using membrane-impermeable amine- or thiol-reactive biotinylation probes as described in Materials and Methods. Surface proteins were collected using Streptavidin-coated beads and analyzed along with the flow-though (internal, non-biotinylated proteins) by immunoblot.

FIG. 7 shows scission of DR5 disulfide bonds triggers high-level DR5 expression, oligomerization, and Caspase 8 activation. FIG. 7A. MDA-MB-468 cells were treated for 24 h with 10 μM dMtcyDTDO or vehicle and cell extracts were immunoprecipitated with ERp44 antibody and analyzed by immunoblot under non-reducing conditions. FIG. 7B. MDA-MB-468 cells ectopically expressing wild type ERp44 or the C58S or S61C ERp44 mutants were treated with 10 μM dMtcyDTDO or vehicle. Left panels, cell extracts were immunoprecipitated with ERp44 antibody and analyzed by immunoblot. Right panels, crude extracts were analyzed for protein expression levels and oligomerization by immunoblot. O and M represent disulfide bonded oligomeric and monomeric protein forms. FIG. 7C. MDA-MB-468 expressing doxycycline-inducible DR4 and DR5 were treated with 1 □g/ml doxycycline, 10 μM dMtcyDTDO, or doxycycline+dMtcyDTDO for 24 h followed by immunoprecipitation of ERp44 and immunoblot analysis of the immunoprecipitates and crude lysates. O and M represent disulfide bonded oligomeric and monomeric protein forms. FIG. 7D. The indicated MDA-MB-468 cell lines stably expressing wild type DR5 or the Δ81-178 mutant lacking part of the disulfide-rich extracellular domain were treated for 24 h as indicated and subjected to immunoprecipitation with ERp44 antibodies. The immunoprecipitates and crude cell lysates were analyzed by non-reducing SDS-PAGE/immunoblot. O and M represent disulfide bonded oligomeric and monomeric protein forms. FIG. 7E. The indicated stable MDA-MB-468 cell lines were treated for 24 h as indicated and subjected to immunoblot analysis after resolving proteins by non-reducing SDS-PAGE. O and M represent disulfide-bonded oligomeric and monomeric protein forms. FIG. 7F. Model for how the biotin-labelled DDA biotin-PyrDTDO reacts covalently with the PDI active site thiolate. Covalent DDA binding is associated with ring-opening and exposure of a nucleophilic sulfinate group, which is capable of attacking the DDA-PDI disulfide bond, resulting in ring closure and DDA excision. It is hypothesized that solvation of the DDA sulfinate group by the PDI slows the rate of DDA self-excision.

FIG. 8 shows models for the molecular mechanisms of DDA action. FIG. 8A. DDAs act on the PDI-family proteins ERp44, AGR2/3, and PDIA1 to alter the disulfide bonding of DR4, DR5, and HER-family proteins. This results in upregulation of DR5, disulfide bond-mediated oligomerization of DR4 and DR5, accumulation of DR4/5 to the cell surface, and activation of the Caspase 8-Caspase 3 pro-apoptotic cascade. Disulfide mediated HER1-3 oligomerization is associated with their downregulation. FIG. 8B. Thioredoxin-like repeat motifs of AGR2, AGR3, and ERp44. FIG. 8C. Model for DDA target protein selectivity in which a thiolate-containing target protein reacts covalently with a DDA, liberating a sulfinate group. Solvation/stabilization of the sulfinate group is hypothesized to prevent DDA release from the protein by recyclization-mediated auto-excision.

FIG. 9 shows characterization of DDAs and an AGR2 DDA binding assay. FIG. 9A. MDA-MB-468 cells were treated for 24 h as indicated and assayed for viability, DNA synthesis, and protein synthesis in MTT assays, and tritiated thymidine and leucine incorporation assays, respectively. FIG. 9B. Reducing immunoblot analysis of MDA-MB-468 cells treated for 24 h as indicated. FIG. 9C. Non-reducing immunoblot analysis of MDA-MB-468 cells treated for 24 h as indicated. FIG. 9D. Immunoblot analysis of MDA-MB-468 and BT474 cells treated for 24 h as indicated. FIGS. 9E and 9F. AGR2 binding assays employing recombinant AGR2 and Biotin-GlyPyrDTDO under the indicated conditions followed by immunoblot analysis for AGR2 or blotting with Streptavidin-conjugated Alkaline Phosphatase.

FIG. 10 shows confirmation of ERp44 and PDIA1 as DDA targets. Peptide coverage of ERp44 and peptide coverage of PDIA1 were identified in Biotin-PyrDTDO/Streptavidin-Agarose pulldowns from MDA-MB-468 cells by tandem mass spectrometry. FIG. 10A. Streptavidin-Agarose pulldowns of Biotin-PyrDTDO treated cells in which the flow through, elution one (250 mM dMtcyDTDO+100 mM 2-Mercaptoethnol), and elution 2 (2× SDS-PAGE Laemmli sample buffer+2 mM Biotin) were analyzed by immunoblot and Streptavidin-Alkaline Phosphatase detection. FIG. 10B. Streptavidin-Agarose pulldowns of Biotin-PyrDTDO treated cells performed in the presence of 100 mM NEM. Half of each sample was brought to 1 M 2-mercaptoethanol and boiled for 20 minutes to generate reduction samples. These non-reducing (left panel) or reducing samples (right panel) were analyzed by blotting with Streptavidin-Alkaline Phosphatase. Molecular weight markers are indicated in kiloDaltons.

### DETAILED DESCRIPTION OF CERTAIN EMBODIMENTS

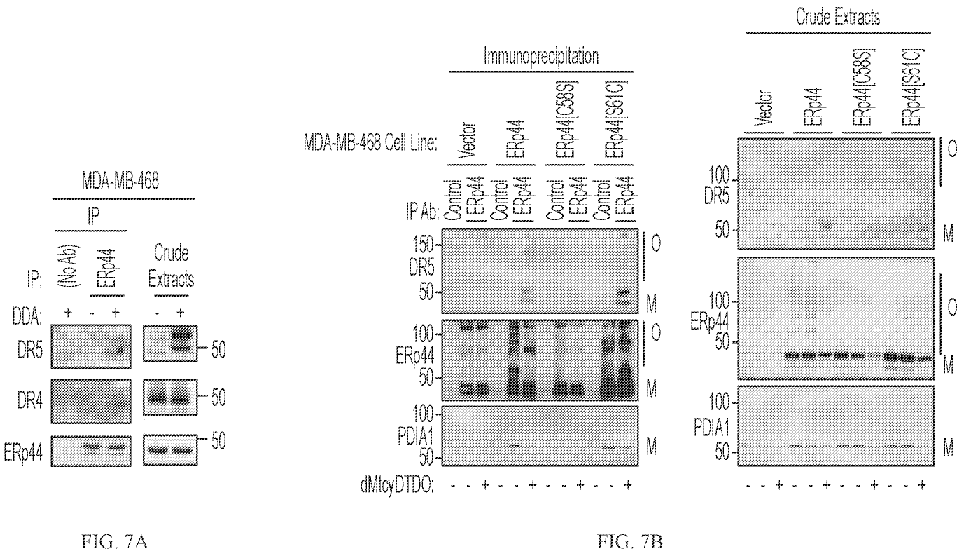

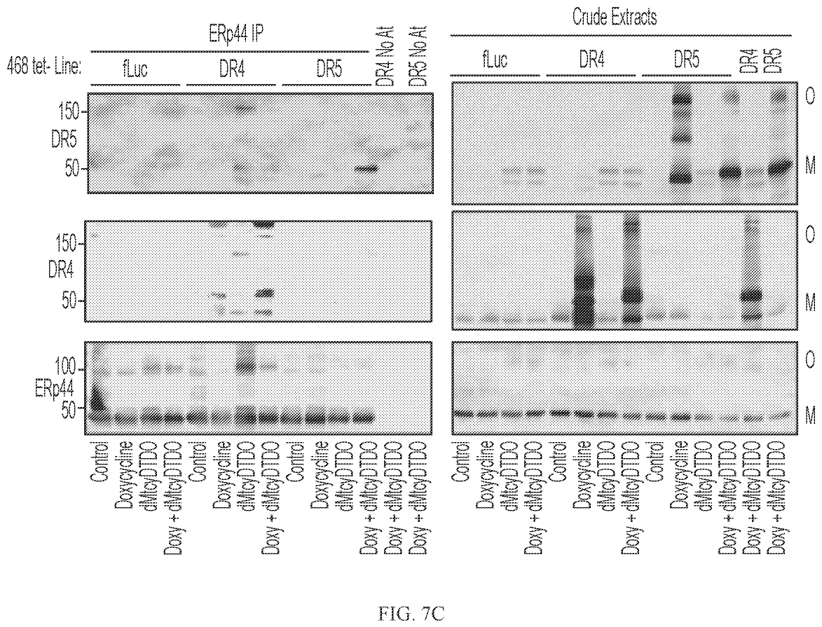

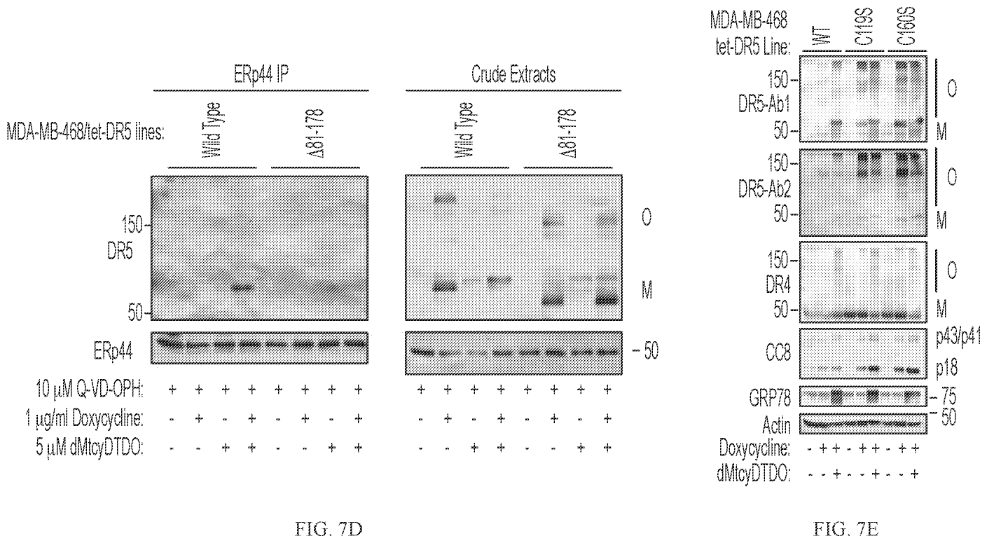

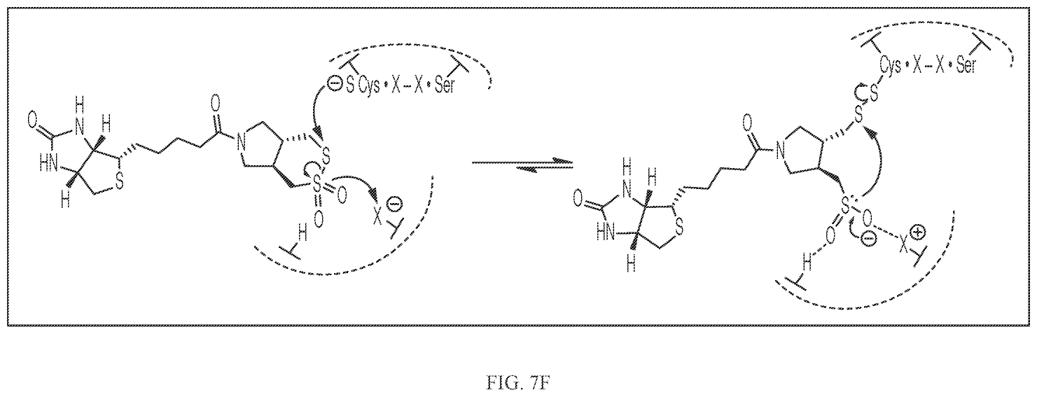

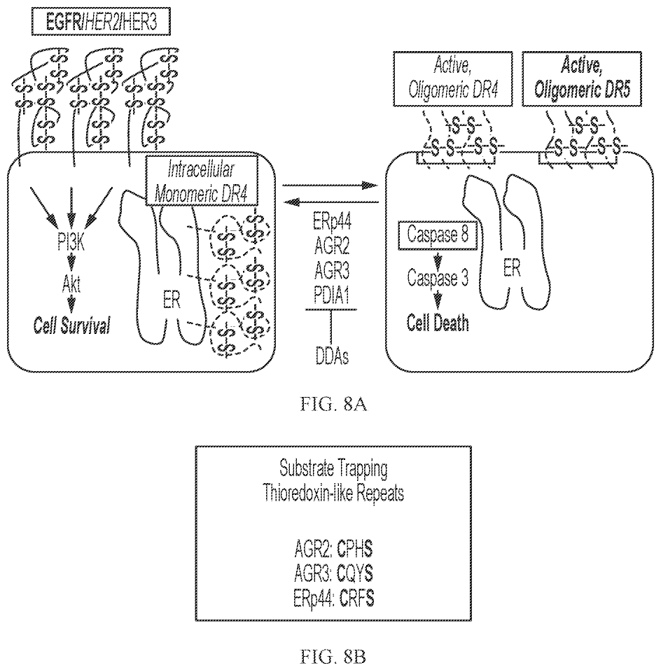

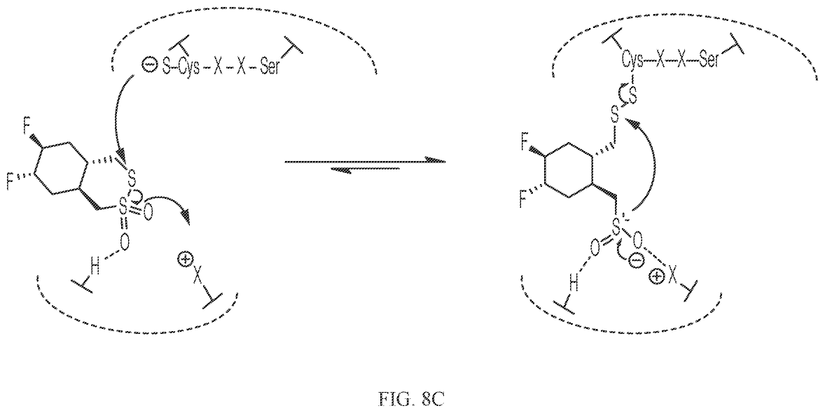

As disclosed herein (e.g., illustrated in FIG. 8A), DDAs bind to the PDI-family proteins ERp44, PDIA1, AGR2, and AGR3 and alter the disulfide bonding of client proteins. These changes in disulfide bonding are associated with upregulation of DR5, oligomerization of DR4 and DR5, and accumulation of DR4 and DR5 at the cell surface. Oligomerization of DR4 and DR5 activates Caspase 8, which in turn activates Caspase 3 to execute apoptosis. Caspase activation plays a key role in downregulating EGFR and HER2, and also in decreasing Akt phosphorylation because these DDA responses are largely ablated by a pan-Caspase inhibitor. DDAs are the first small molecule agents that target ERp44 and AGR2/3. Further, DDAs are the first experimental anticancer agents identified that activate DR4 and DR5-mediated Caspase 8 activation by inducing disulfide bond-mediated DR4/5 clustering in the absence of their ligand TRAIL. The extracellular domain of DR5 acts to prevent its oligomerization in the absence of TRAIL. DDAs may overcome this autoinhibition by favoring intermolecular disulfide bonding between DR4/5 extracellular domains over intramolecular disulfide bonding. Consistent with these interpretations, mutational disruption of individual DR5 disulfide bonds is sufficient to trigger DR5 upregulation, oligomerization, and downstream activation of Caspase 8 (FIG. 7E).

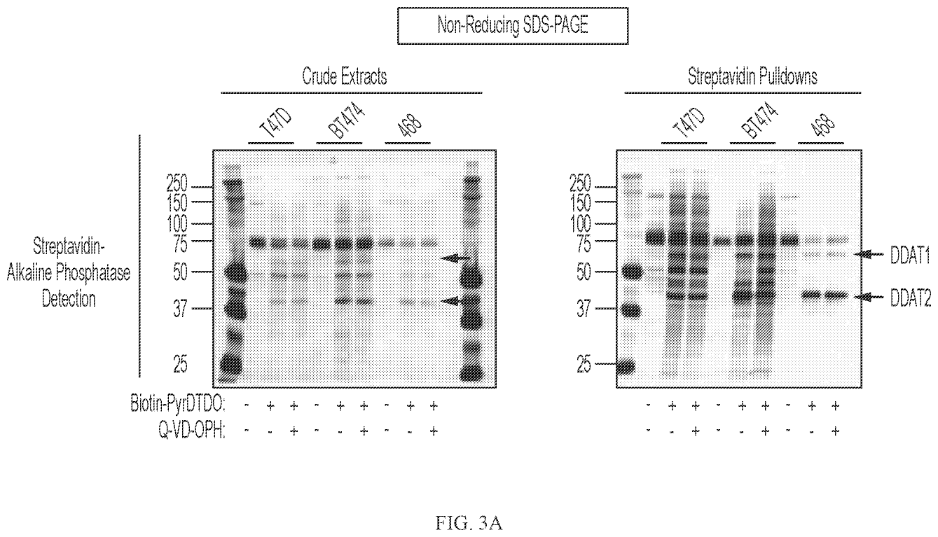

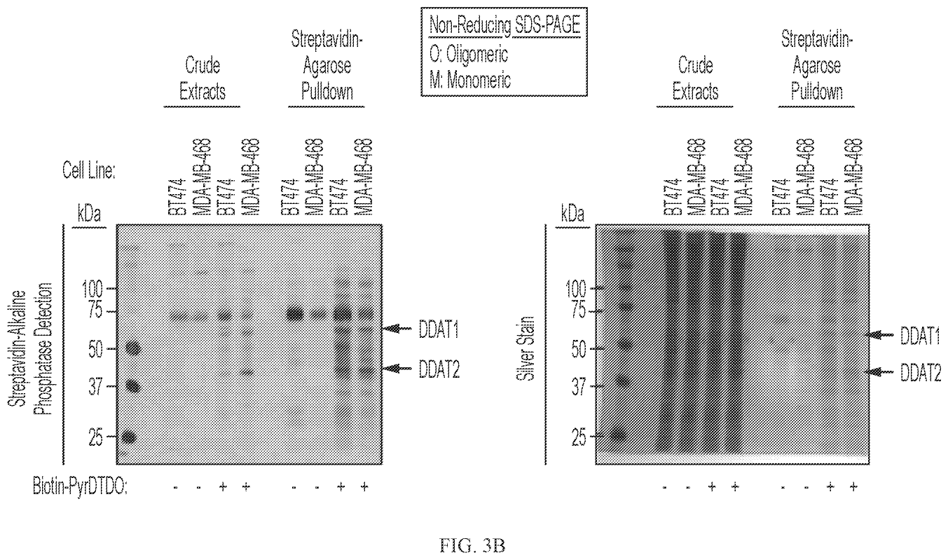

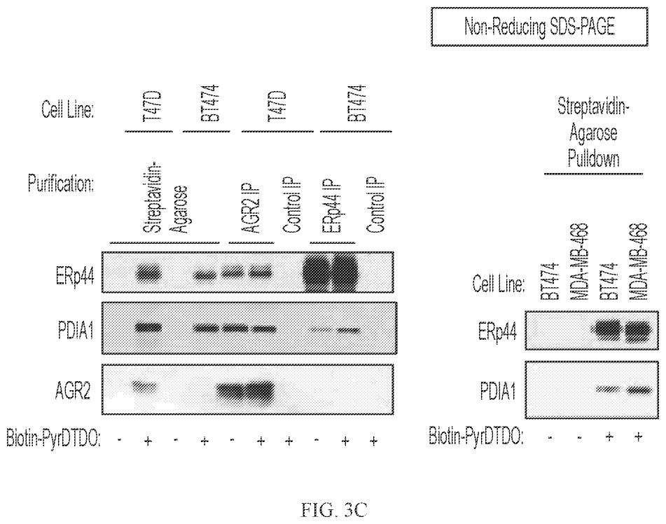

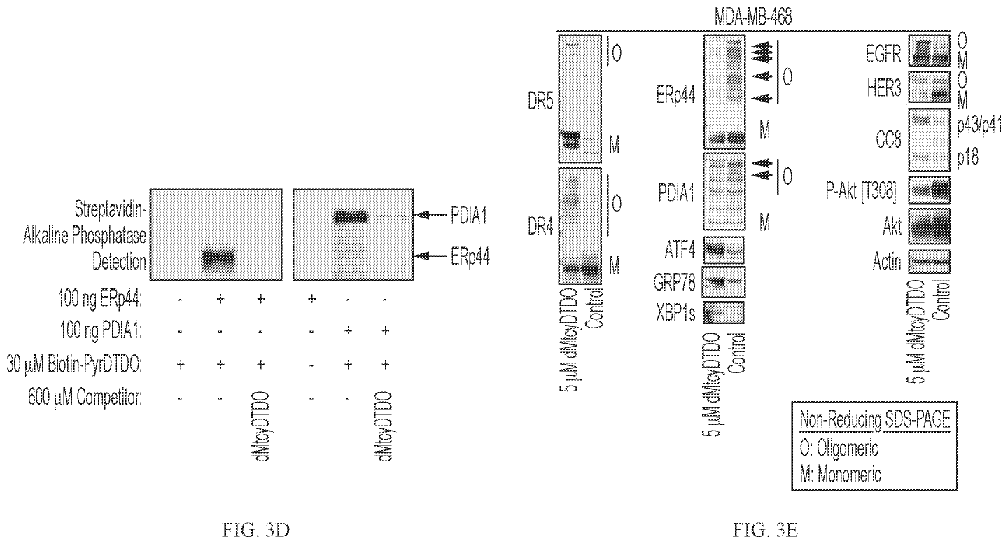

Overexpression of EGFR amplifies multiple DDA responses, including ERS induction, DR5 upregulation, HER1-3 downregulation, and reduction of Akt phosphorylation. Discovery of ERp44 and PDIA1 as DDA target proteins led to the observation that EGFR overexpression reduces the levels of monomeric ERp44 and PDIA1 accessible for facilitating native disulfide bonding of client proteins. Since DDAs covalently modify monomeric ERp44 and PDIA1 (FIG. 3), these observations reveal the molecular basis for the synthetic-lethal interaction between EGFR overexpression and DDA-based therapies. Given the importance of EGFR as a potential oncogenic driver in a number of cancers, including glioblastoma and Triple-Negative Breast Cancer, DDAs may have utility against these malignancies.

Because of the relative structural simplicity of DDA molecules, the mechanisms responsible for their target protein selectivity are of interest. PDIA1 has been investigated as a target for anticancer therapeutics, and PDIA1 inhibitors have established anticancer efficacy in preclinical models. AGR2 contributes to tumor progression by facilitating EGFR disulfide bonding and surface presentation. However, AGR2 may also be secreted and promote tumor angiogenesis and act on cell surface receptors. Monoclonal antibodies designed to block extracellular AGR2 functions are under development, but agents that block the essential role of AGR2 in the folding of client proteins are not available.

The canonical thioredoxin-like catalytic site sequence CXXC is featured in PDIA1 and several other PDI enzymes. Interestingly, the DDA targets AGR2, AGR3, and ERp44 share a non-canonical CXXS thioredoxin-like repeat sequence (FIG. 8B). A report employing both yeast genetics and biochemical enzyme assays showed that the essential function of the PDI enzyme for yeast survival is catalysis of disulfide exchange or “scramblase” activity and that this could be carried out by the CXXS mutant of PDI (PDI^{CXXS}). Enzyme assays showed that PDI^{CXXS} could reactivate inappropriately disulfide bonded RNAse A via its scramblase activity. In contrast, PDI^{CXXS} was unable to catalyze disulfide bond oxidation or reduction like the wild type PDI^{CXXC} enzyme.

The C-terminal Cys residue in the CXXC motif of PDIs accelerates substrate release. Consequently, CXXS substrate-trapping mutants of PDIs have been used to isolate mixed disulfide bonded dimers with their client/substrate proteins. That the CXXS motif is associated with slow client protein off-rate is consistent with the established roles of ERp44 in retaining specific client/partner proteins such as ERO1 and ERAP1 in the ER, and in retrotranslocation of inappropriately disulfide bonded Adiponectin and IgM from the Golgi to the ER. Thus, the CXXS motifs of AGR2, AGR3, and ERp44 bound to DDAs may exhibit a slow off-rate for the same reason.

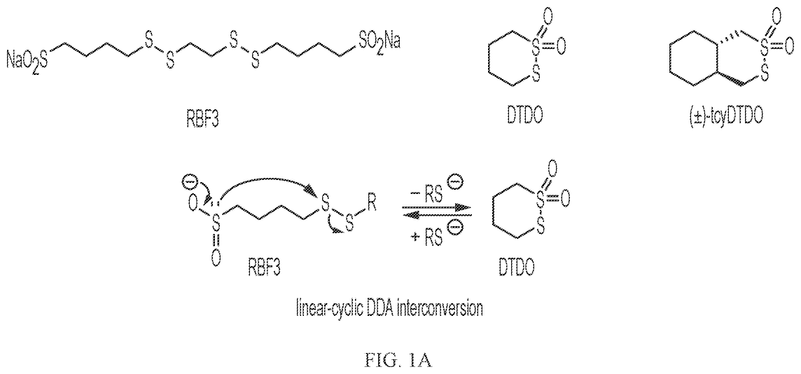

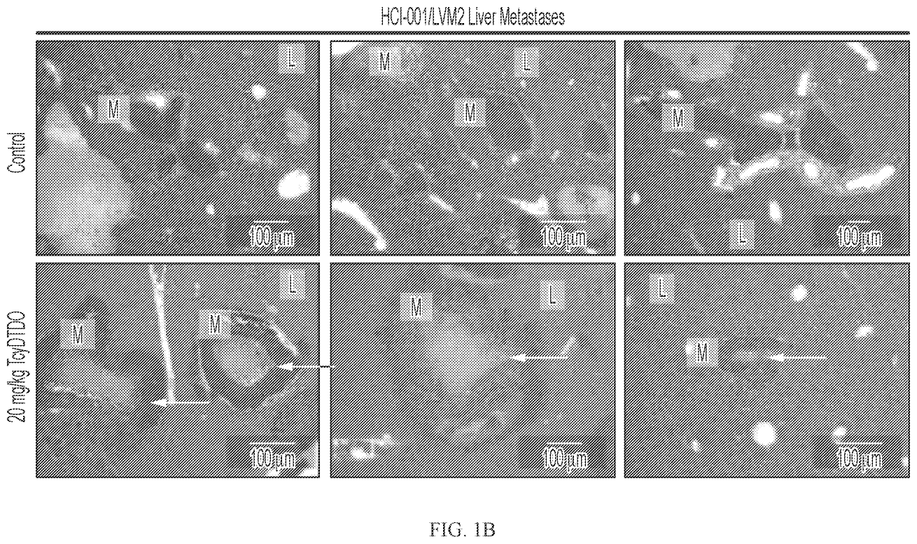

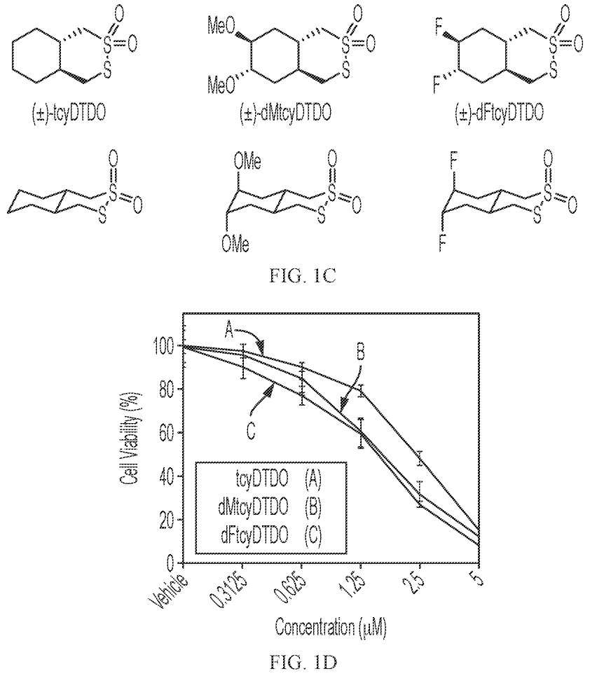

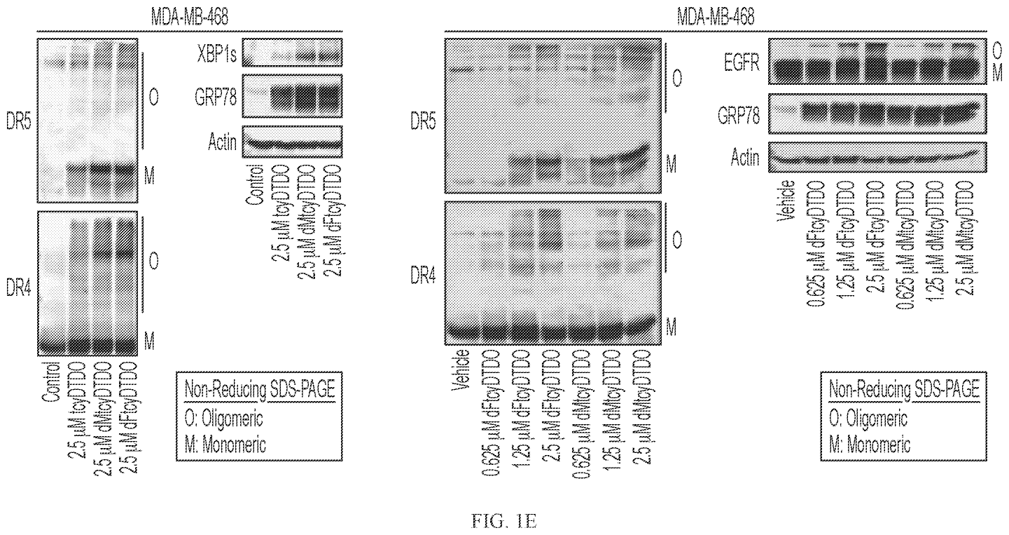

The DDA pharmacophore has the capacity to adopt cyclic or linear structures (FIG. 1). Reaction of a protein-associated Cysteine thiolate nucleophile with the electrophilic sulfenyl sulfur atom on the thiosulfonate moiety of cyclic DDAs opens the ring and generates the linear DDA form containing a free nucleophilic sulfinate group that can mount an attack on the disulfide bond to regenerate the cyclic DDA and excise itself from the protein (acting as reversible covalent ring opening/closing inhibitors) (FIG. 7E, 8C). Thus, the proteins targeted by DDAs are predicted to be those that stabilize the linear form of the DDAs by appropriate noncovalent interactions to slow the corresponding back reaction (cyclization and auto-excision).

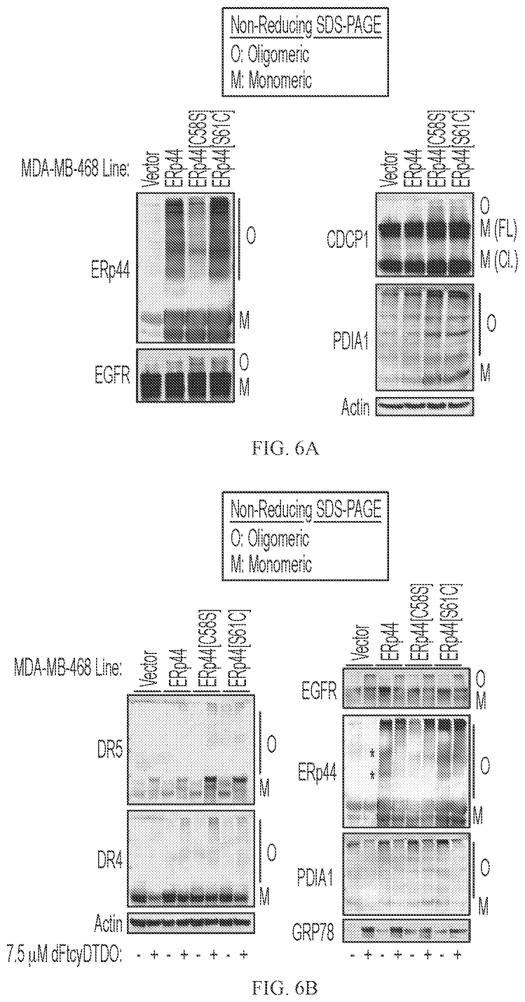

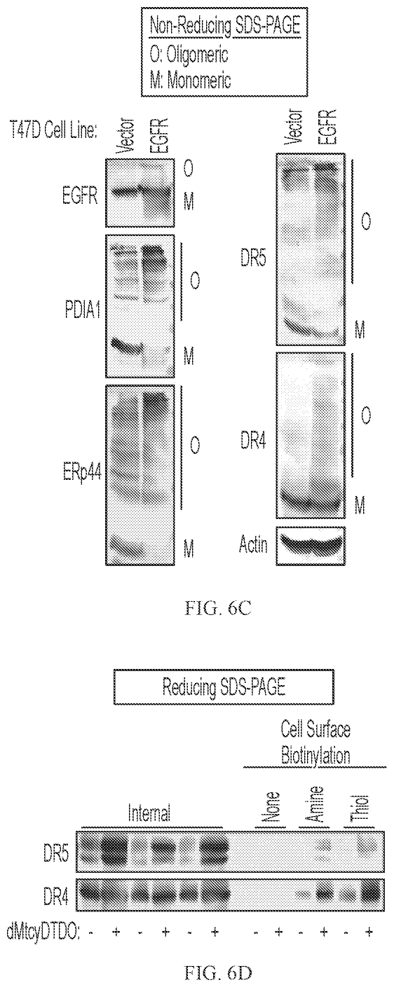

Disclosed herein DDAs alter the disulfide bonding of DR4, DR5, and EGFR and change the pattern of disulfide bonding between ERp44 and PDIA1 and their client proteins. DDA selectivity for killing cancer cells and for stably binding to its target proteins derives from the following combination of factors: 1) EGFR or HER2 overexpression in cancer cells limits the pool of free ERp44 and PDIA1 available to mediate disulfide bonding of clients such as DR4, DR5, and EGFR itself; 2) Some potential DDA targets may have the capacity to excise DDAs using Cysteine residues adjacent to the DDA-modified Cysteine; and 3) DDAs have the ability to auto-excise by cyclization, therefore target proteins must stabilize the linear form of the bound DDA. Interest in covalent protein targeting drugs is increasing because temporal target engagement is less limited by their pharmacokinetic properties than non-covalent therapeutics. DDAs exemplify how covalent drugs may be utilized to kill cancer cells with minimal effects on normal cells. Further, the observation that tumor sensitivity to DDAs will vary inversely with the levels of monomeric DDA target proteins (e.g., FIG. 6C) allow for the development of predictive diagnostic tests of tumor biopsies that identify the patients that are most likely to benefit from DDA-based treatment regimens.

### Definitions

Before further description of the present disclosure, and in order that the disclosure may be more readily understood, certain terms are first defined and collected here for convenience.

The term “administration” or “administering” includes routes of introducing the compound of the disclosure to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the disclosure can be coated with or disposed in a selected material to protect it from natural conditions which may detrimentally affect its ability to perform its intended function. The compound of the disclosure can be administered alone, or in conjunction with either another agent as described above or with a pharmaceutically acceptable carrier, or both. The compound of the disclosure can be administered prior to the administration of the other agent, simultaneously with the agent, or after the administration of the agent. Furthermore, the compound of the disclosure can also be administered in a pro-drug form which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In certain embodiments, the term “lower alkyl” includes a straight chain alkyl having 4 or fewer carbon atoms in its backbone, e.g., C1-C4 alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The term “haloalkoxy” as used herein, refers to the radical of —O-alkyl substituted with 1-5 halo atoms (e.g., fluoromethoxy, trifluoromethoxy, pentafluoroethoxy, and the like). The alkyl can be straight chain or branched chain containing 1-10 carbon atoms. Halo atoms independently are F, Cl, Br, or I, which can be the same or different in a haloalkoxy moiety.

The terms “alkenyl” and “alkynyl” refer to unsaturated aliphatic groups analogous in length and possible substitution to the alkyls described above, but that contain at least one double or triple bond, respectively. For example, the disclosure contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” of a compound of the disclosure includes all activities elicited by compound of the disclosure in a responsive cell. It includes genomic and non-genomic activities elicited by these compounds.

“Biological composition” or “biological sample” refers to a composition containing or derived from cells or biopolymers. Cell-containing compositions include, for example, mammalian blood, red cell platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In one embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In certain embodiments, the blood cell suspension includes red blood cells and/or platelets and/or leukocytes and/or bone marrow cells.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient to treat a cell proliferative disorder. An effective amount of compound of the disclosure may vary according to factors such as the disease state, age, and weight of the subject, and the ability of the compound of the disclosure to elicit a desired response in the subject. Dosage regimens may be adjusted to provide the optimum therapeutic response. An effective amount is also one in which any toxic or detrimental effects (e.g., side effects) of the compound of the disclosure are outweighed by the therapeutically beneficial effects.

A therapeutically effective amount of compound of the disclosure (i.e., an effective dosage) may range from about 0.001 to 30 mg/kg body weight, or about 0.01 to 25 mg/kg body weight, or about 0.1 to 20 mg/kg body weight, or about 1 to 10 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the disclosure can include a single treatment or can include a series of treatments. In one example, a subject is treated with a compound of the disclosure in the range of between about 0.1 to 20 mg/kg body weight, one time per week for between about 1 to 10 weeks, or between 2 to 8 weeks, or between about 3 to 7 weeks, or for about 4, 5, or 6 weeks. It will also be appreciated that the effective dosage of a compound of the disclosure used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “improved biological properties” refers to any activity inherent in a compound of the disclosure that enhances its effectiveness in vivo. In certain embodiments, this term refers to any qualitative or quantitative improved therapeutic property of a compound of the disclosure, such as reduced toxicity.

The term “cell proliferative disorder” includes disorders involving the undesired or uncontrolled proliferation of a cell. Examples of such disorders include, but are not limited to, tumors (e.g., brain, lung (small cell and non-small cell), ovary, prostate, breast or colon) or other carcinomas or sarcomas (e.g., leukemia, lymphoma).

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, oxo, halogen, cyano, nitro, C₁-C₈alkyl, C₂-C₈alkenyl, C₂-C₈alkynyl, C₁-C₈alkoxy, C₂-C₈alkyl ether, C₃-C₈alkanone, C₁-C₈alkylthio, amino, mono- or di-(C1-C₈alkyl)amino, haloC₁-C₈alkyl, haloC₁-C₈alkoxy, C₁-C₈alkanoyl, C₂-C₈alkanoyloxy, C₁-C₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrases “substituted or unsubstituted’ and “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substituents).

The term “isomers” or “stereoisomers” refers to compounds which have identical chemical constitution, but differ with regard to the arrangement of the atoms or groups in space.

The term “modulate” refers to an increase or decrease, e.g., in the ability of a cell to proliferate in response to exposure to a compound of the disclosure, e.g., the inhibition of proliferation of at least a sub-population of cells in an animal such that a desired end result is achieved, e.g., a therapeutic result.

The term “obtaining” as in “obtaining a compound capable of inhibiting CDCP1” is intended to include purchasing, synthesizing or otherwise acquiring the compound.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” or “pro-drug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound of the disclosure any formula herein or otherwise described herein which is effective, upon single or multiple dose administration to the patient, in preventing or treating a cell proliferative disorder.

The language “reduced toxicity” is intended to include a reduction in any undesired side effect elicited by a compound of the disclosure when administered in vivo.

The term “sulfhydryl” or “thiol” means —SH.

The term “subject” includes organisms which are capable of suffering from a cell proliferative disorder or who could otherwise benefit from the administration of a compound of the disclosure, such as human and non-human animals. Preferred humans include human patients suffering from or prone to suffering from a cell proliferative disorder or associated state, as described herein. The term “non-human animals” of the disclosure includes all vertebrates, e.g., mammals; e.g., rodents; e.g., mice; and non-mammals, such as non-human primates; e.g., sheep, dog, cow, chickens, amphibians, reptiles, etc.

The term “susceptible to a cell proliferative disorder” is meant to include subjects at risk of developing disorder of cell proliferation, e.g., cancer, i.e., subjects suffering from viral infection with cancer causing viruses, subjects that have been exposed to ionizing radiation or carcinogenic compounds, subjects having a family or medical history of cancer, and the like.

The phrases “systemic administration,” “administered systemically”, “peripheral administration” and “administered peripherally” as used herein mean the administration of a compound of the disclosure, drug or other material, such that it enters the patient's system and, thus, is subject to metabolism and other like processes, for example, subcutaneous administration.

The language “therapeutically effective amount” of a compound of the disclosure refers to an amount of an agent which is effective, upon single or multiple dose administration to the patient, in inhibiting cell proliferation and/or symptoms of a cell proliferative disorder, or in prolonging the survivability of the patient with such a cell proliferative disorder beyond that expected in the absence of such treatment.

The terms “oligomer”, as used herein, refers to a protein comprising more than one repeating unit (“monomer”) of that protein, wherein the repeating units are associated with each other, e.g., by a chemical bond (e.g., a disulfide bond linking monomers of the protein). In certain embodiments, the oligomer comprises at least two repeating units, i.e., the monomer. The “oligomeric form” is language denoting identification of the oligomer in its form of multiple monomeric repeat units of the protein associated. In certain embodiments, oligomeric form includes a sum of all the different oligomers that may be present at the time oligomeric forms are measured.

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

### Methods

In one aspect, disclosed herein is a method of determining the ratio of oligomeric forms of one of ERp44, PDIA1, or AGR2 relative to the monomeric form of that same protein in a biological sample of a subject, the method comprising: (a) measuring the amount of oligomeric forms of one of ERp44, PDIA1, or AGR2 in the subject sample; (b) measuring the amount of monomeric form of the same protein measured in step (a) in the subject sample; and (c) comparing the measurement of (a) and the measurement of (b) to determine the ratio of oligomeric forms to monomeric form.

In certain embodiments, the amount measured is a mass unit. In certain embodiments, the amount measured is a molar unit.

In certain embodiments, the method comprises determining the ratio of oligomeric forms of ERp44 or PDIA1 relative to the monomeric form of that same protein in a biological sample of a subject, the method comprising: (a) measuring the amount of oligomeric forms of one of ERp44 or PDIA1 in the subject sample; (b) measuring the amount of monomeric form of the same protein measured in step (a) in the subject sample; and (c) comparing the measurement of (a) and the measurement of (b) to determine the ratio of oligomeric forms to monomeric form.

In certain embodiments, the method comprises determining the ratio of oligomeric forms of ERp44. In certain embodiments, the method comprises determining the ratio of oligomeric forms of PDIA1. In certain embodiments, the method comprises determining the ratio of oligomeric forms of AGR2.

In certain embodiments, the measuring comprises non-reducing immunoblot analysis of subject tumor tissue samples.

In certain embodiments, the method further comprises reporting the result of step (c).

In certain embodiments, if the ratio determined in step (c) is greater than a ratio of measurements in (a)/(b) taken from a healthy subject sample, then the subject is identified as in need of cancer therapy.

In certain embodiments, if the ratio determined in step (c) is greater than or equal to 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.5, 3.0., 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 100, 200, 300, 400, 500, 1000, 10000, 100000, or 1000000, then the subject is identified as in need of cancer therapy.

In certain embodiments, if the ratio determined in step (c) is 0.05-1.0, 1.0-10, 10-100, 100-1000, 1000-10000, 10000-100000, or 100000-1000000, then the subject is identified as in need of cancer therapy. In certain embodiments, if the ratio determined in step (c) is 0.05-0.25, 0.25-0.5, 0.5-0.75, 0.75-1.0, 1.0-10, 10-100, 100-1000, 1000-10000, 10000-100000, or 100000-1000000, then the subject is identified as in need of cancer therapy.

In certain embodiments, the ratio can be expressed as a percentage of oligomerization of the protein (ERp44, PDIA1, or AGR2).

In another aspect, disclosed herein is a method of lowering the ratio of oligomeric forms of one of ERp44, PDIA1, or AGR2 relative to the monomeric form of that same protein in a biological sample of a subject, the method comprising administering a compound to the subject, wherein the subject is determined to have a high ratio of oligomeric forms of one of ERp44, PDIA1, or AGR2 relative to the monomeric form of that same protein.

In certain embodiments, the method comprises lowering the ratio of oligomeric forms of one of ERp44 or PDIA1 relative to the monomeric form of that same protein in a biological sample of a subject, the method comprising administering a compound to the subject, wherein the subject is determined to have a high ratio of oligomeric forms of one of ERp44 or PDIA1 relative to the monomeric form of that same protein.

In certain embodiments, the method comprises lowering the ratio of oligomeric forms of ERp44 relative to the monomeric form of that same protein. In certain embodiments, the method comprises lowering the ratio of oligomeric forms of PDIA1 relative to the monomeric form of that same protein. In certain embodiments, the method comprises lowering the ratio of oligomeric forms of AGR2 relative to the monomeric form of that same protein.

In certain embodiments, the compound is an inhibitor of ERp44, PDIA1, or AGR2. In certain embodiments, the compound is an inhibitor of ERp44 or PDIA1. In certain embodiments, the compound is an inhibitor of ERp44. In certain embodiments, the compound is an inhibitor of PDIA1. In certain embodiments, the compound is an inhibitor of AGR2.

In certain embodiments, a high ratio of oligomeric forms to monomeric form is a ratio greater than a ratio of oligomeric forms to monomeric form of that same protein taken from a healthy subject sample. In certain embodiments, the healthy subject sample is of a different subject than the subject of the method. In certain embodiments, the healthy subject sample is of the same subject of the method. In certain embodiments, the healthy subject sample is of the same subject of the method and was taken at a time when the subject may not have been suffering from cancer.

In certain embodiments, a high ratio of oligomeric forms to monomeric form is a ratio greater than or equal to 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.5, 3.0., 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 100, 200, 300, 400, 500, 1000, 10000, 100000, or 1000000.

In certain embodiments, a high ratio of oligomeric forms to monomeric form is a ratio of 0.05-1.0, 1.0-10, 10-100, 100-1000, 1000-10000, 10000-100000, or 100000-1000000. In certain embodiments, a high ratio of oligomeric forms to monomeric form is a ratio of 0.05-0.25, 0.25-0.5, 0.5-0.75, 0.75-1.0, 1.0-10, 10-100, 100-1000, 1000-10000, 10000-100000, or 100000-1000000.

In certain embodiments, the ratio can be expressed as a percentage of oligomerization of the protein (ERp44, PDIA1, or AGR2).

In another aspect, disclosed is a method of treating a patient comprising administering a cancer therapy to the patient, wherein the patient is determined to have a high ratio of oligomeric forms of one of ERp44, PDIA1, or AGR2 relative to the monomeric form of that same protein.

In certain embodiments, the patient is determined to have a high ratio of oligomeric forms of one of ERp44 or PDIA1 relative to the monomeric form of that same protein. In certain embodiments, the patient is determined to have a high ratio of oligomeric forms of one of ERp44 relative to the monomeric form of that same protein. In certain embodiments, the patient is determined to have a high ratio of oligomeric forms of PDIA1 relative to the monomeric form of that same protein. In certain embodiments, the patient is determined to have a high ratio of oligomeric forms of AGR2 relative to the monomeric form of that same protein.

In certain embodiments, the cancer therapy is an inhibitor of ERp44, PDIA1, or AGR2. In certain embodiments, the cancer therapy is an inhibitor of ERp44 or PDIA1. In certain embodiments, the cancer therapy is an inhibitor of ERp44. In certain embodiments, the cancer therapy is an inhibitor of PDIA1. In certain embodiments, the cancer therapy is an inhibitor of AGR2.

In certain embodiments, a high ratio of oligomeric forms to monomeric form is a ratio greater than a ratio of oligomeric forms to monomeric form of that same protein taken from a healthy subject sample. In certain embodiments, the healthy subject sample is of a different subject than the subject of the method. In certain embodiments, the healthy subject sample is of the same subject of the method. In certain embodiments, the healthy subject sample is of the same subject of the method and was taken at a time when the subject may not have been suffering from cancer.

In certain embodiments, a high ratio of oligomeric forms to monomeric form is a ratio greater than or equal to 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.5, 3.0., 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 100, 200, 300, 400, 500, 1000, 10000, 100000, or 1000000.

In certain embodiments, a high ratio of oligomeric forms to monomeric form is a ratio of 0.05-1.0, 1.0-10, 10-100, 100-1000, 1000-10000, 10000-100000, or 100000-1000000.

In certain embodiments, the cancer is glioblastoma, pancreatic cancer, or breast cancer. In certain embodiments, the cancer is breast cancer. In certain embodiments, is HER2-positive breast cancer. In certain embodiments, is triple-negative breast cancer.

In certain embodiments, the subject is identified as having a cancer expected to have enhanced response to therapy by administration of an inhibitor of ERp44, PDIA1, or AGR2. In certain embodiments, the subject is identified as having a cancer expected to have enhanced response to therapy by administration of an inhibitor of ERp44 or PDIA1. In certain embodiments, the subject is identified as having a cancer expected to have enhanced response to therapy by administration of an inhibitor of ERp44. In certain embodiments, the subject is identified as having a cancer expected to have enhanced response to therapy by administration of an inhibitor of PDIA1. In certain embodiments, the subject is identified as having a cancer expected to have enhanced response to therapy by administration of an inhibitor of AGR2.

In certain embodiments, the cancer therapy is chemotherapy. Exemplary chemotherapeutic agents include, but are not limited to, anti-estrogens (e.g., tamoxifen, raloxifene, and megestrol), LHRH agonists (e.g., goscrclin and leuprolide), anti-androgens (e.g., flutamide and bicalutamide), photodynamic therapies (e.g., vertoporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., ‘2’-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonuclotide reductase inhibitors (e.g., hydroxyurea and deferoxamine), uracil analogs (e.g., 5-fluorouracil (5-FU), floxuridine, doxifluridine, ratitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In certain embodiments, the compound or cancer therapy is a DDA. In certain embodiments, the DDA is a compound of Formula (I), Formula (II), or Formula (III). In certain embodiments, the DDA is a compound of Formula (I). In certain embodiments, the DDA is a compound of Formula (II). In certain embodiments, the DDA is a compound of Formula (III).

In another aspect, disclosed herein is a test panel comprising an agent to assess the presence of ERp44 and/or an agent to assess the presence of PDIA1 in a subject sample. In certain embodiments, the test panel comprises an agent to assess the presence of ERp44 and an agent to assess the presence of PDIA1 in a subject sample. In certain embodiments, the test panel further comprises an agent to assess the presence of AGR2 or AGR3. In certain embodiments, the test panel further comprises an agent to assess the presence of one or more of EGFR, HER2, HER3, DR4, and DR5.

In certain embodiments, the agent is a small molecule or an antibody. In certain embodiments, the agent is a detectably labelled small molecule or a detectably labelled antibody. In certain embodiments, the agent is a detectably labelled DDA.

In certain embodiments, the detectably labelled DDA is a compound of Formula (I), (II), or (III). In certain embodiments, the detectably labelled DDA is a compound of Formula (III).

In another aspect, disclosed herein is a method of testing for the presence of ERp44 and/or PDIA1 in a subject sample comprising contacting the test panel described herein with a subject sample. In certain embodiments, the method comprises testing for the presence of ERp44 and PDIA1 in a subject sample comprising contacting the test panel with a subject sample. In certain embodiments, the method comprises testing for the presence of oligomeric and monomeric forms of ERp44 and/or PDIA1 in a subject sample comprising contacting the test panel with a subject sample. In certain embodiments, the method comprises testing for the presence of oligomeric and monomeric forms of ERp44 in a subject sample comprising contacting the test panel with a subject sample. In certain embodiments, the method comprises testing for the presence of oligomeric and monomeric forms of PDIA1 in a subject sample comprising contacting the test panel with a subject sample.

In certain embodiments, the sample is tissue, cell, blood, saliva, sputum, serum, or plasma.

In certain embodiments, the method further comprises testing for the presence of AGR2 or AGR3 in the sample. In certain embodiments, the method comprises reporting the presence or absence of AGR2 or AGR3 in the sample.

In certain embodiments, the method comprises reporting the presence or absence of ERp44 and/or PDIA1 in the subject sample. In certain embodiments, the method comprises reporting the presence or absence of ERp44 in the subject sample. In certain embodiments, the method comprises reporting the presence or absence of PDIA1 in the subject sample.

In certain embodiments, the method comprises reporting the presence or absence of oligomeric and monomeric forms of ERp44 and/or PDIA1 in the subject sample. In certain embodiments, the method comprises reporting the presence or absence of oligomeric and monomeric forms of ERp44 in the subject sample. In certain embodiments, the method comprises reporting the presence or absence of oligomeric and monomeric forms of PDIA1 in the subject sample.

In certain embodiments, the method comprises testing for the presence of one or more of EGFR, HER2, HER3, DR4, and DR5 in the sample. In certain embodiments, the method comprises reporting the presence or absence of one or more of EGFR, HER2, HER3, DR4, and DR5 in the sample.

In certain embodiments, the disclosure provides methods for treating a subject for a cell proliferative disorder, by administering to the subject an effective amount of a compound of Formula (I)-(III), or salt thereof; or a pharmaceutical composition comprising a compound of Formula (I)-(III) or salt thereof. A cell proliferative disorder includes cancer. In certain embodiments, the subject is a mammal, e.g., a primate, e.g., a human.

A further aspect presents a method of treating a subject suffering from or susceptible to cancer, including administering to the subject an effective amount of a compound of any one of Formulae (I)-(III), or salt, solvate, hydrate or prodrug thereof; or a pharmaceutical composition thereof, to thereby treat the subject suffering from or susceptible to cancer.

In certain embodiments, the aforementioned methods of the disclosure include administering to a subject a therapeutically effective amount of a compound of any of the formulae herein (e.g., Formula (I)-(III)), or salt thereof, in combination with another pharmaceutically active compound. Examples of pharmaceutically active compounds include compounds known to treat cell proliferative disorders, e.g., breast cancer therapeutics; pancreatic cancer therapeutics, etc. Other pharmaceutically active compounds that may be used include antibodies to AGR2, ERp44, and/or PDIA1. Exemplary DR5 antibodies include, but are not limited to, conatumumab, drozitumab, apomab, DAB4, PRO95780, lexatumumab, HGS-ETR2, tigatuzumab, CS-1008, TRA-8, HGSTR2J, KMTRS, LBY-135, and the like. Other pharmaceutically active compounds that may be used include a DR5 agonist, including TRAIL or TRAIL analog (e.g., TRAIL 10 trimer, stabilized form of TRAIL, and the like), a DR5 agonist antibody, or a TRAIL synthesis inducer (e.g., TIC10), all of which are described in WO 2019/241644 (published Dec. 19, 2019; incorporated by reference). The compound of the disclosure and the pharmaceutically active compound may be administered to the subject in the same pharmaceutical composition or in different pharmaceutical compositions (at the same time or at different times).

In certain embodiments, the aforementioned methods of the disclosure include administering to a subject a therapeutically effective amount of a compound of any of the formulae herein (e.g., Formula (I)-(III)), or salt thereof, in combination with another conventional treatment regimen known in the art. Conventional treatment regimens for cancer and for other tumors include radiation, surgery, drugs, or combinations thereof.

Determination of a therapeutically effective anti-proliferative amount or a prophylactically effective anti-proliferative amount of the compound of the disclosure of the disclosure, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective anti-proliferative amount or dose, and the prophylactically effective anti-proliferative amount or dose, a number of factors are considered by the attending clinician, including, but not limited to: the specific cell proliferative disorder involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the disclosure with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective anti-proliferative amount of a compound of the disclosure of the disclosure is expected to vary from about 0.1 milligram per kilogram of body weight per day (mg/kg/day) to about 100 mg/kg/day.

Compounds determined to be effective for the prevention or treatment of cell proliferative disorders in animals, e.g., dogs, chickens, and rodents, may also be useful in treatment of tumors in humans. Those skilled in the art of treating tumors in humans will know, based upon the data obtained in animal studies, the dosage and route of administration of the compound to humans. In general, the dosage and route of administration in humans is expected to be similar to that in animals.

The identification of those patients who are in need of prophylactic treatment for cell proliferative disorders is well within the ability and knowledge of one skilled in the art. Certain of the methods for identification of patients which are at risk of developing cell proliferative disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

A method of assessing the efficacy of a treatment in a subject includes determining the pre-treatment extent of a cell proliferative disorder by methods well known in the art (e.g., determining tumor size or screening for tumor markers where the cell proliferative disorder is cancer) and then administering a therapeutically effective amount of an inhibitor of cell proliferation (e.g., a compound of any formula herein or otherwise described herein) according to the disclosure to the subject. After an appropriate period of time after the administration of the compound (e.g., 1 day, 1 week, 2 weeks, one month, six months), the extent of the cell proliferative disorder is determined again. The modulation (e.g., decrease) of the extent or invasiveness of the cell proliferative disorder indicates efficacy of the treatment. The extent or invasiveness of the cell proliferative disorder may be determined periodically throughout treatment. For example, the extent or invasiveness of the cell proliferative disorder may be checked every few hours, days or weeks to assess the further efficacy of the treatment. A decrease in extent or invasiveness of the cell proliferative disorder indicates that the treatment is efficacious. The method described may be used to screen or select patients that may benefit from treatment with an inhibitor of a cell proliferative disorder.

As used herein, “obtaining a biological sample from a subject,” includes obtaining a sample for use in the methods described herein. A biological sample is described above.

In another aspect, a compound of any of the formulae herein (e.g., Formula (I)-(III)), or salt thereof, or pharmaceutical composition comprising a compound of any of the formulae herein (e.g., Formula (I)-(III)) or salt thereof is packaged in a therapeutically effective amount with a pharmaceutically acceptable carrier or diluent. The composition may be formulated for treating a subject suffering from or susceptible to a cell proliferative disorder and packaged with instructions to treat a subject suffering from or susceptible to a cell proliferative disorder.

In another aspect, methods of inhibiting a cell proliferative disorder in a subject include administering an effective amount of a compound of the disclosure to the subject. The administration may be by any route of administering known in the pharmaceutical arts. The subject may have a cell proliferative disorder, may be at risk of developing a cell proliferative disorder, or may need prophylactic treatment prior to anticipated or unanticipated exposure to conditions capable of increasing susceptibility to a cell proliferative disorder, e.g., exposure to carcinogens or to ionizing radiation.

In one aspect, a method of monitoring the progress of a subject being treated includes determining the pre-treatment status (e.g., size, growth rate, or invasiveness of a tumor) of the cell proliferative disorder, administering a therapeutically effective amount of a compound of any of the formulae herein (e.g., Formulae (I)-(III)), or salt, solvate, hydrate or prodrug thereof, (or a dosing regimen or pharmaceutical composition thereof) to the subject, and determining the status of the cell proliferative disorder after an initial period of treatment, wherein the modulation of the status indicates efficacy of the treatment.

The subject may be at risk of a cell proliferative disorder, may be exhibiting symptoms of a cell proliferative disorder, may be susceptible to a cell proliferative disorder and/or may have been diagnosed with a cell proliferative disorder.

The initial period of treatment may be the time in which it takes to establish a stable and/or therapeutically effective blood serum level of any of the compounds, compound combinations, dosing regimens, or pharmaceutical compositions delineated herein, or the time in which it take for the subject to clear a substantial portion of the compound, or any period of time selected by the subject or healthcare professional that is relevant to the treatment.

If the modulation of the status indicates that the subject may have a favorable clinical response to the treatment, the subject may be treated with the compound. For example, the subject can be administered a therapeutically effective dose or doses of the compound.

In another aspect, the disclosure provides methods for inhibiting EGFR, HER2, and/or HER3 signaling in a cell. The methods include contacting the cell with an effective amount of any of the compounds, compound combinations, dosing regimens, or pharmaceutical compositions delineated herein, such that the signaling of EGFR, HER2, and/or HER3 is reduced. The contacting may be in vitro, e.g., by addition of the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

The EGFR, HER2, and/or HER3 may be within a cell, isolated from a cell, recombinantly expressed, purified or isolated from a cell or recombinant expression system or partially purified or isolated from a cell or recombinant expression system.

The contacting may be in vitro, e.g., by addition of the compound to a solution containing purified EGFR, HER2, and/or HER3, or, if EGFR, HER2, and/or HER3 is present in cells, by adding the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

Kits of the disclosure include kits for treating a cell proliferative disorder in a subject. The disclosure also includes kits for downregulating expression of EGFR, HER2, and/or HER3, stabilizing an interaction of EGFR, HER2, and/or HER3, assessing the efficacy of a treatment for a cell proliferative disorder in a subject, monitoring the progress of a subject being treated for a cell proliferative disorder, selecting a subject with a cell proliferative disorder for treatment according to the disclosure, and/or treating a subject suffering from or susceptible to a cell proliferative disorder. The kit may include any of the compounds, compound combinations, dosing regimens, or pharmaceutical compositions delineated herein and instructions for use. The instructions for use may include information on dosage, method of delivery, storage of the kit, etc. The kits may also include reagents, for example, test compounds, buffers, media (e.g., cell growth media), cells, etc. Test compounds may include known compounds or newly discovered compounds, for example, combinatorial libraries of compounds. One or more of the kits of the disclosure may be packaged together, for example, a kit for assessing the efficacy of a treatment for a cell proliferative disorder may be packaged with a kit for monitoring the progress of a subject being treated for a cell proliferative disorder according to the disclosure.

The present methods can be performed on cells in culture, e.g. in vitro or ex vivo, or on cells present in an animal subject, e.g., in vivo. Compounds of the disclosures can be initially tested in vitro using primary cultures of proliferating cells, e.g., transformed cells, tumor cell lines, and the like.

Alternatively, the effects of compound of the disclosure can be characterized in vivo using animal models.

### Compounds

In one aspect, the disclosure provides a compound (e.g., any compound of a formulae herein) that inhibits or is capable of inhibiting AGR2, ERp44 and/or PDIA1.

In one aspect, the disclosure provides a pharmaceutical composition that inhibits or is capable of inhibiting AGR2, ERp44 and/or PDIA1, the pharmaceutical composition comprising: 1) a compound of any of the formula herein (i.e., any of Formula I-III), or salt, solvate, hydrate or prodrug thereof.

Disulfide bond Disrupting Agents (DDAs) are a class of anticancer compounds that selectively kill cancers that overexpress the Epidermal Growth Factor Receptor (EGFR) or its family member HER2. DDAs kill EGFR+ and HER2+ cancer cells via the parallel downregulation of EGFR, HER2, and HER3 and activation/oligomerization of Death Receptors 4 and 5 (DR4/5). Examples of DDAs and their preparation include those described in, for example, PCT/US2018/018979, PCT/US2019/037209, and PCT/US2021/022542, all incorporated herein by reference.

In certain embodiments, the compound (DDA) is of Formula (I):

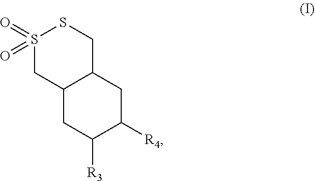

or a salt, solvate, hydrate or prodrug thereof, wherein:
(i) R₃ is halo or haloalkoxy; and R₄ is halo or haloalkoxy; or
(ii) R₃ is H or C₁-C₆ alkoxy; and R₄ is halo or haloalkoxy; or
(iii) R₃ is halo or haloalkoxy; and R₄ is H or C₁-C₆ alkoxy.

In certain embodiments, the compound (DDA) is of Formula (I-a), (I-b), (I-c), (I-d), (I-e), (I-f), (I-g), (I-h), (I-i), or (I-j):

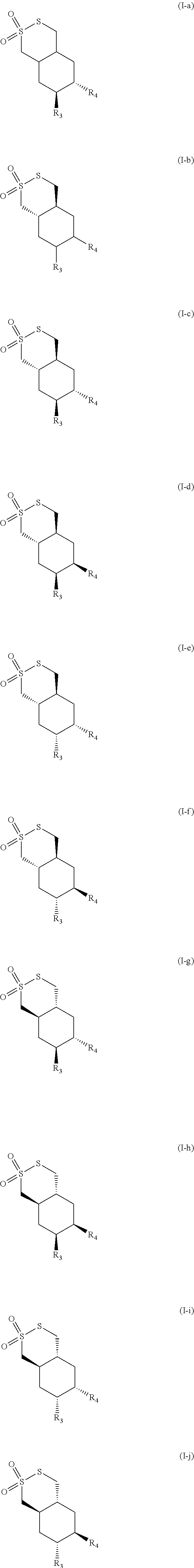

or a salt, solvate, hydrate or prodrug thereof.

In certain embodiments, the compound (DDA) is any of the following compounds:

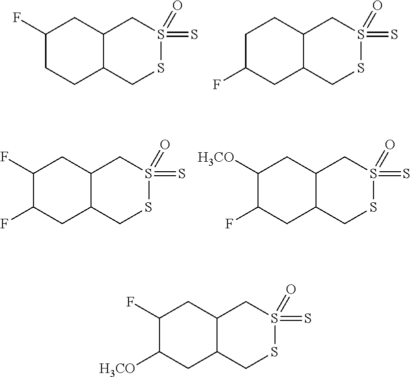 [SMILES: O=S1(=S)CC2CC(F)CCC2CS1 ; O=S1(=S)CC2CCC(F)CC2CS1 ; O=S1(=S)CC2CC(F)C(F)CC2CS1 ; COC1CC2CS(=O)(=S)SCC2CC1F ; COC1CC2CSS(=O)(=S)CC2CC1F]

or a salt, solvate, hydrate or prodrug thereof.

In certain embodiments, the compound (DDA) is any of the following compounds:

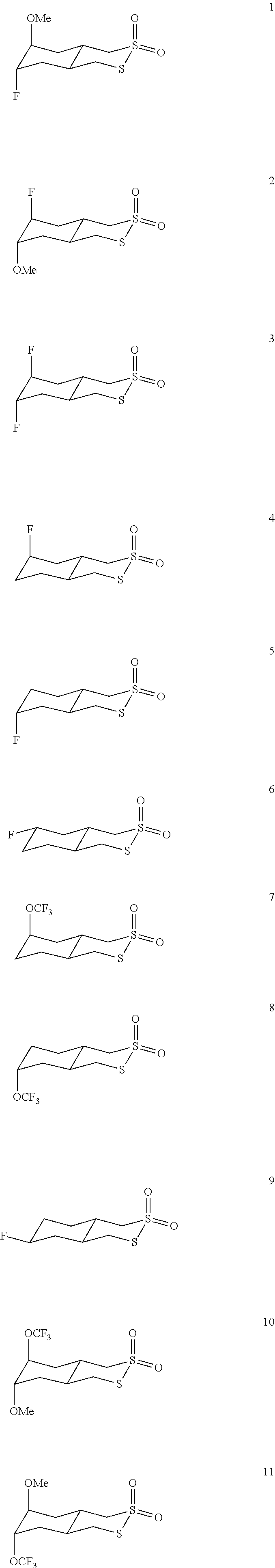

or a salt, solvate, hydrate or prodrug thereof.

In certain embodiments, the compound (DDA) is any of the following compounds:

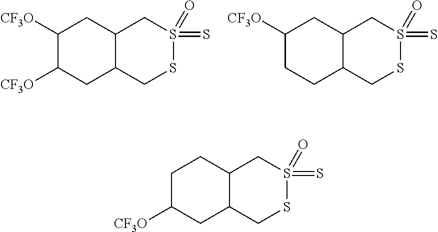

or a salt, solvate, hydrate or prodrug thereof.

In certain embodiments, the compound (DDA) is a compound of formula (II):

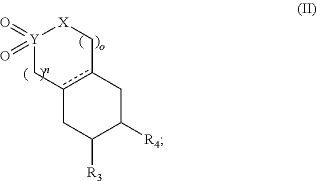

or a salt, solvate, hydrate or prodrug thereof, wherein:
X is S or Se;
Y is S or Se;
R₃ is selected from H or C₁-C₆ alkoxy;
R₄ is selected from H or C₁-C₆ alkoxy;
n is 0, 1, 2, or 3;
o is 0, 1, 2, or 3; and
[symbol] denotes a carbon-carbon single bond or double bond;
wherein if [symbol] is a single bond, X and Y are both S, and n and o are each 1, then at least one of R₃ or R₄ is C₁-C₆ alkoxy.

The following embodiments pertain to compounds of Formula (I) and/or (II):

In certain embodiments, R₃ is halo or haloalkoxy; and R₄ is halo or haloalkoxy. In some embodiments, R₃ is halo; and R₄ is halo. In some embodiments, R₃ is fluoro; and R₄ is fluoro. In certain embodiments, R₃ is haloalkoxy; and R₄ is haloalkoxy. In certain embodiments, R₃ is —OCF₃; and R₄ is —OCF₃. In some embodiments, R₃ is haloalkoxy; and R₄ is halo. In some embodiments, R₃ is —OCF₃; and R₄ is halo. In some embodiments, R₃ is haloalkoxy; and R₄ is fluoro. In certain embodiments, R₃ is halo; and R₄ is haloalkoxy. In certain embodiments, R₃ is halo; and R₄ is —OCF₃. In certain embodiments, R₃ is fluoro; and R₄ is haloalkoxy.

In some embodiments, R₃ is H or C₁-C₆ alkoxy; and R₄ is halo or haloalkoxy. In some embodiments, R₃ is H; and R₄ is halo. In some embodiments, R₃ is H; and R₄ is fluoro. In certain embodiments, R₃ is H; and R₄ is haloalkoxy. In certain embodiments, R₃ is H; and R₄ is —OCF₃. In some embodiments, R₃ is C₁-C₆ alkoxy; and R₄ is halo. In some embodiments, R₃ is methoxy; and R₄ is halo. In some embodiments, R₃ is C₁-C₆ alkoxy; and R₄ is fluoro. In certain embodiments, R₃ is C₁-C₆ alkoxy; and R₄ is haloalkoxy. In certain embodiments, R₃ is C₁-C₆ alkoxy; and R₄ is —OCF₃. In certain embodiments, R₃ is methoxy; and R₄ is haloalkoxy.

In certain embodiments, R₃ is halo or haloalkoxy; and R₄ is H or C₁-C₆ alkoxy. In some embodiments, R₃ is halo or haloalkoxy; and R₄ is H. In some embodiments, R₃ is halo or —OCF₃; and R₄ is H. In some embodiments, R₃ is fluoro or haloalkoxy; and R₄ is H. In some embodiments, R₃ is halo or —OCF₃; and R₄ is H. In certain embodiments, R₃ is halo or haloalkoxy; and R₄ is C₁-C₆ alkoxy. In certain embodiments, R₃ is halo or haloalkoxy; and R₄ is methoxy. In certain embodiments, R₃ is fluoro or haloalkoxy; and R₄ is C₁-C₆ alkoxy. In certain embodiments, R₃ is halo or —OCF₃; and R₄ is C₁-C₆ alkoxy.

In some embodiments, haloalkoxy is fluoro-C₁-C₆-alkoxy. In some embodiments, haloalkoxy is fluoro-C₁-C₃-alkoxy, and C₁-C₆ alkoxy is C₁-C₃-alkoxy. In some embodiments, haloalkoxy is —OCF₃ and C₁-C₆ alkoxy is C₁-C₃-alkoxy. In some embodiments, haloalkoxy is —OCF₃ and C₁-C₆ alkoxy is methoxy. In some embodiments, halo is fluoro, haloalkoxy is —OCF₃, and C₁-C₆ alkoxy is methoxy.

In certain embodiments, the compound (DDA) is a compound of formula (III):

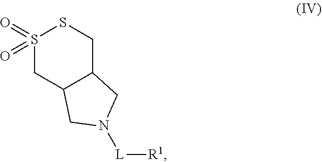

or a salt, solvate, hydrate or prodrug thereof, wherein:
L is a bond, substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —C(═O)O—, or —OC(═O)N(R^{A})—, or a combination thereof;
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or two R^{A} groups are joined to form a substituted or unsubstituted heterocyclic ring; and
R¹ is hydrogen, a nitrogen protecting group, or a label.

In certain embodiments, L is substituted or unsubstituted alkylene, substituted or unsubstituted heteroalkylene, —C(═O)NR^{A}—, —C(═O)—, —NR^{A}C(═O)R^{A}—, —C(═O)R^{A}—, or —NR^{A}C(═O)—; or a combination thereof. In certain embodiments, L is substituted or unsubstituted alkylene, —C(═O)NR^{A}—, —C(═O)—, or a combination thereof. In certain embodiments, L is a combination of substituted or unsubstituted alkylene, —C(═O)NR^{A}—, and —C(═O)—. In certain embodiments, L is a combination of substituted or unsubstituted alkylene and —C(═O)—.

In certain embodiments, L is

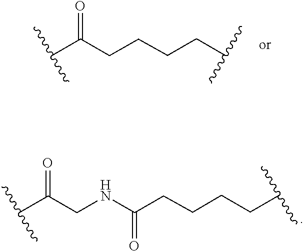 [SMILES: *CCCCC(*)=O ; *CCCCC(=O)NCC(*)=O]

In certain embodiments, L is

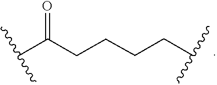 [SMILES: *CCCCC(*)=O]

In certain embodiments, L is

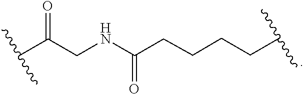 [SMILES: *CCCCC(=O)NCC(*)=O]

In certain embodiments, R¹ is hydrogen.

In certain embodiments, R¹ is a nitrogen protecting group. In certain embodiments, the nitrogen protecting group is selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N ,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-l-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate. In certain embodiments, the nitrogen protecting group is Bn, Boc, Cbz, Fmoc, trifluoroacetyl, triphenylmethyl, acetyl, or Ts. In certain embodiments, the nitrogen protecting group is Boc.

In certain embodiments, R¹ is a label. In certain embodiments, R¹ is a label capable of providing a detectable signal, either directly or indirectly. Exemplary labels include radioisotopes, fluorescent molecules, isotopes (e.g., radioactive isotopes), biotin, and the like. In certain embodiments, R¹ is biotin.

In certain embodiments, the compound (DDA) is a compound of any of the following formula:

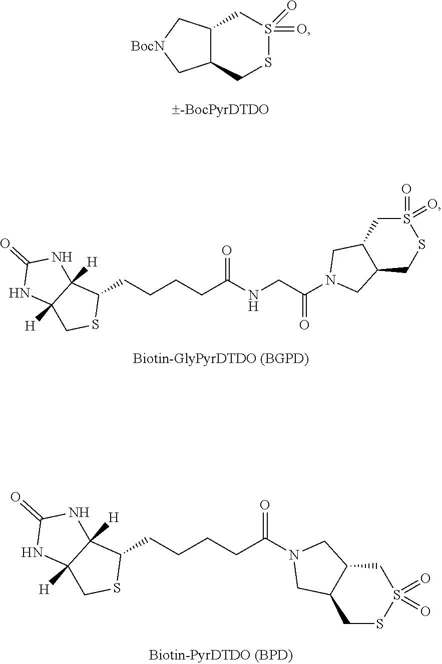 [SMILES: CC(C)(C)OC(=O)N1C[C@@H]2CS(=O)(=O)SC[C@H]2C1 ; O=C(CCCC[C@@H]1SC[C@@H]2NC(=O)N[C@@H]21)NCC(=O)N1C[C@@H]2CS(=O)(=O)SC[C@H]2C1 ; O=C1N[C@H]2[C@H](CS[C@H]2CCCCC(=O)N2C[C@@H]3CS(=O)(=O)SC[C@H]3C2)N1]

or a salt, solvate, hydrate or prodrug thereof.

In certain embodiments, the compound (DDA) is a compound of formula:

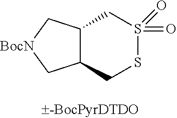 [SMILES: CC(C)(C)OC(=O)N1C[C@@H]2CS(=O)(=O)SC[C@H]2C1]

or a salt, solvate, hydrate or prodrug thereof.

In certain embodiments, the compound (DDA) is a compound of formula:

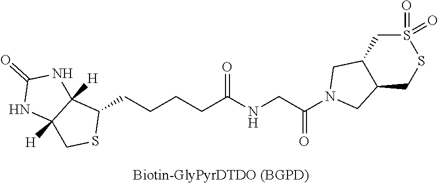 [SMILES: O=C(CCCC[C@@H]1SC[C@@H]2NC(=O)N[C@@H]21)NCC(=O)N1C[C@@H]2CS(=O)(=O)SC[C@H]2C1]

or a salt, solvate, hydrate or prodrug thereof.

In certain embodiments, the compound (DDA) is a compound of formula:

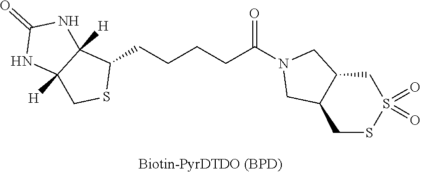 [SMILES: O=C1N[C@H]2[C@H](CS[C@H]2CCCCC(=O)N2C[C@@H]3CS(=O)(=O)SC[C@H]3C2)N1]

or a salt, solvate, hydrate or prodrug thereof.

When preparing compounds of the disclosure, naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

Definitions of specific functional groups and chemical terms are described herein. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers. While compounds may be depicted as racemic or as one or more diastereoisomers, enantiomers, or other isomers, all such racemic, diastereoisomer, enantiomer, or other isomer forms of that depicted are included in the present disclosure.

### Pharmaceutical Compositions

In another aspect, the disclosure provides a pharmaceutical composition comprising: a compound of any of the formulae herein (e.g., Formula (I)-(III)), or salt, solvate, hydrate or prodrug thereof; and a pharmaceutically acceptable carrier.

In another aspect, the disclosure provides a method for making a pharmaceutical composition comprising combining: a compound of any of the formulae herein (e.g., Formula (I)-(III)), or salt, solvate, hydrate or prodrug thereof; and a pharmaceutically acceptable carrier.

In an embodiment, the compound of the disclosure is administered to the subject using a pharmaceutically-acceptable formulation, e.g., a pharmaceutically-acceptable formulation that provides sustained delivery of the compound of the disclosure to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compounds of the disclosure, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, oxalic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present. Solubilizing agents, including for example, cremaphore and beta-cyclodextrins can also be used in the pharmaceutical compositions herein.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The disclosure also provides a pharmaceutical composition, comprising an effective amount of a compound described herein and a pharmaceutically acceptable carrier. In an embodiment, compound is administered to the subject using a pharmaceutically acceptable formulation, e.g., a pharmaceutically acceptable formulation that provides sustained delivery of the compound to a subject for at least 12 hours, 24 hours, 36 hours, 48 hours, one week, two weeks, three weeks, or four weeks after the pharmaceutically acceptable formulation is administered to the subject.

By “pharmaceutically effective amount” as used herein is meant an amount of a compound of the disclosure, high enough to significantly positively modify the condition to be treated but low enough to avoid serious side effects (at a reasonable benefit/risk ratio), within the scope of sound medical judgment. A pharmaceutically effective amount of a compound of the disclosure will vary with the particular goal to be achieved, the age and physical condition of the patient being treated, the severity of the underlying disease, the duration of treatment, the nature of concurrent therapy and the specific compound employed. For example, a therapeutically effective amount of a compound of the disclosure administered to a child or a neonate will be reduced proportionately in accordance with sound medical judgment. The effective amount of a compound of the disclosure will thus be the minimum amount which will provide the desired effect.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the disclosure include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 per cent to about ninety-nine percent of active ingredient, or from about 5 per cent to about 70 per cent, or from about 10 per cent to about 30 per cent.

Methods of preparing these compositions include the step of bringing into association a compound of the disclosure with the carrier and, optionally, one or more accessory ingredients. In general, the formulations are prepared by uniformly and intimately bringing into association a compound of the disclosure with liquid carriers, or finely divided solid carriers, or both, and then, if necessary, shaping the product.

Compositions of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the disclosure as an active ingredient. A compound may also be administered as a bolus, electuary or paste.

In solid dosage forms of the disclosure for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the disclosure include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the disclosure may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the disclosure for rectal or vaginal administration may be presented as a suppository, which may be prepared by mixing one or more compound of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present disclosure which are suitable for vaginal administration also include pessaries, tampons, creams, gels, pastes, foams or spray formulations containing such carriers as are known in the art to be appropriate.

Dosage forms for the topical or transdermal administration of a compound of the disclosure include powders, sprays, ointments, pastes, creams, lotions, gels, solutions, patches and inhalants. The active compound of the disclosure may be mixed under sterile conditions with a pharmaceutically acceptable carrier, and with any preservatives, buffers, or propellants which may be required.

The ointments, pastes, creams and gels may contain, in addition to compound of the disclosure of the present disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the disclosure can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the disclosure to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the disclosure.

Pharmaceutical compositions of the disclosure suitable for parenteral administration comprise one or more compound of the disclosure in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of compound of the disclosure in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the disclosure is administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Regardless of the route of administration selected, the compound of the disclosure, which may be used in a suitable hydrated form, and/or the pharmaceutical compositions of the present disclosure, are formulated into pharmaceutically acceptable dosage forms by conventional methods known to those of skill in the art.

Actual dosage levels and time course of administration of the active ingredients in the pharmaceutical compositions of the disclosure may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the compound of the disclosure for the present disclosure is the maximum that a patient can tolerate and not develop serious side effects. Preferably, the compound of the disclosure of the present disclosure is administered at a concentration of about 0.001 mg to about 100 mg per kilogram of body weight.

For nasal administration or administration by inhalation or insufflation, the active compound(s) or prodrug(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

A specific example of an aqueous suspension formulation suitable for nasal administration using commercially available nasal spray devices includes the following ingredients: active compound or prodrug (0.5-20 mg/ml); benzalkonium chloride (0.1-0.2 mg/mL); polysorbate 80 (TWEEN® 80; 0.5-5 mg/ml); carboxymethylcellulose sodium or microcrystalline cellulose (1-15 mg/ml); phenylethanol (1-4 mg/ml); and dextrose (20-50 mg/ml). The pH of the final suspension can be adjusted to range from about pH 5 to pH 7, with a pH of about pH 5.5 being typical.

For prolonged delivery, the active compound(s) or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in for example, U.S. Pat. Nos. 5,407,713; 5,352,456; 5,332,213; 5,336,168; 5,290,561; 5,254,346; 5,164,189; 5,163,899; 5,088,977; 5,087,240; 5,008,110; and 4,921,475, each of which is incorporated herein by reference in its entirety.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) also can be employed.

The pharmaceutical compositions can, if desired, be presented in a pack or dispenser device which can contain one or more unit dosage forms containing the active compound(s). The pack can, for example, comprise metal or plastic foil, such as a blister pack. The pack or dispenser device can be accompanied by instructions for administration.

The active compound(s) or prodrug(s) of the presently disclosed subject matter, or compositions thereof, will generally be used in an amount effective to achieve the intended result, for example in an amount effective to treat or prevent the particular disease being treated. The compound(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. Therapeutic benefit also includes halting or slowing the progression of the disease, regardless of whether improvement is realized.

For prophylactic administration, the compound can be administered to a patient at risk of developing one of the previously described diseases. A patient at risk of developing a disease can be a patient having characteristics placing the patient in a designated group of at risk patients, as defined by an appropriate medical professional or group. A patient at risk may also be a patient that is commonly or routinely in a setting where development of the underlying disease that may be treated by administration of an enzyme inhibitor according to the disclosure could occur. In other words, the at risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC50 of the particular compound as measured in an in vitro assay, such as those described in the Examples section. Calculating dosages to achieve such circulating blood or serum concentrations taking into account the bioavailability of the particular compound is well within the capabilities of skilled artisans. For guidance, see Goodman and Gilman's The Pharmacological Basis of Therapeutics, 13th or latest edition, McGraw Hill Medical, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) which are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

The compound(s) can be administered once per day, a few or several times per day, or even multiple times per day, depending upon, among other things, the indication being treated and the judgment of the prescribing physician.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

Another object of the present disclosure is the use of a compound as described herein (e.g., of any formulae herein) in the manufacture of a medicament for use in the treatment of a disorder or disease (e.g., any disorder or disease herein). Another object of the present disclosure is the use of a compound as described herein (e.g., of any formulae herein) for use in the treatment of a disorder or disease (e.g., any disorder or disease herein).

### EXAMPLES

The disclosure is further illustrated by the following examples which are intended to illustrate but not limit the scope of the disclosure.

### Cell Culture, Preparation of Cell Extracts, and Immunoblot Analysis

The BT20, BT474, BT549, MCF7, MDA-MB-231, MDA-MB-436, MDA-MB-468, HCC1143, HCC1500, HCC1569, HCC1937, HCC1954, Hs578t, and T47D cells lines were purchased from American Type Culture Collection (ATCC) (Manassas, VA). The HCI-001 cell line was derived from an ER-/PR-/HER2- patient-derived xenograft originally isolated from a patient as described previously (DeRose Y S, et al. Nat Med 2011; 17: 1514-1520). The HCI-001/LVM2 model was derived from the HCI-001 cell line by performing two rounds of selection for liver metastasis, as described previously (Wang M, et al. Cell Death Discovery 2019; 5: 153). Cell lines were grown in Dulbecco's Modified Eagle's Medium (GE Healthcare Life Sciences, Logan, UT) containing 10% fetal bovine serum (10% FBS—DMEM) in a humidified incubator with 5% CO₂ at 37° C. Cell lysates were prepared as described previously (Law B K, et al. Mol Cell Biol. 2002; 22: 8184-8198). Immunoblot analysis was performed utilizing the following antibodies purchased from Cell Signaling Technology (Beverly, MA) [Akt, #4691; P-Akt[T308], #13038; ATF4, #11815; CHOP, #2895; Cleaved Caspase 8, #9496; DR4, #42533; DR5, #8074; EGFR, #4267; GRP78, #3177; HER3, #4754; PARP, #9532; PDIA1, #3501; XBP1s, #12782], from Santa Cruz Biotechnology (Santa Cruz, CA) [Actin, sc-47778; AGR2/3, sc-376653; AGR3, sc-390940; ERO1-a, sc-100805; and ERp44, sc-393687], or from Rockland Immunochemicals, Inc. (Limerick, PA), Streptavidin-Alkaline Phosphatase conjugated (SA-AP), S000-05.

### Construction of Vectors and Stable Cell Lines Using Recombinant Retroviruses and Lentiviral shRNAs

Total RNA from T47D cells was extracted with TRIzol Reagent (Invitrogen, Waltham, MA) according to the manufacturer's protocol. Total cellular RNA was used to synthesize first-strand cDNA by using the PCR conditions listed: 25° C. for 10 min, 42° C. for 30 min, and 95° C. for 5 min. PCR was subsequently performed using the following ERp44 primers: 5′-TTTTGGATCCCACCATGCATCCTGCCGTCTTCC-3′ and 5′-TTTTCTCGAGTTAAAGCTCATCTCGATCCCTC-3′. The PCR fragment encoding ERp44 was subsequently digested with BamHI and XhoI and cloned into the 5′ BamHI and 3′ XhoI sites of the pMXs-IRES-Blasticidin retroviral vector (RTV-016) (Cell Biolabs, Inc., San Diego, CA). The C58S and S61C mutations of Erp44 were performed using QuikChange mutagenesis and the following primers: 5′-CTTTAGTAAATTTTTATGCTGACTGGAGTCGTTTCAGTCAGATGTTG-3′ and 5′-CAACATCTGACTGAAACGACTCCAGTCAGCATAAAAATTTACTAAAG-3′ and 5′-GACTGGTGTCGTTTCTGTCAGATGTTGCATCCAATTTTTG-3′ and 5′-CAAAAATTGGATGCAACATCTGACAGAAACGACACCAGTC-3′, respectively. Mutation of ERp44 was verified by sequencing. The retroviral vector encoding EGFR (Plasmid 11011) was purchased from Addgene (Cambridge, MA). Stable cell lines were constructed as described previously (Law M E, et al. Breast Cancer Res 2016; 18: 80; Law M E, et al. Oncogene 2013; 32: 1316-1329). The ERp44 shRNA lentiviral constructs were obtained from the TRC Lentiviral shRNA Libraries from Thermo Scientific. Stable cell lines were constructed from these lentiviral shRNAs according to vendor protocol.

### Thymidine Incorporation Assays

Thymidine incorporation assays were performed as described previously (Corsino P, et al. J Biol Chem 2009; 284: 29945-29955).

### Protein Synthesis Assays

Protein synthesis assays utilizing ³H-Leucine purchased from Perkin Elmer (Waltham, MA) were carried out as detailed previously (Law B K, et al. J Biol Chem 2000; 275: 10796-10801; Law B K, et al. J Biol Chem 2000; 275: 38261-38267).

### Cell Viability Assays

MTT (3-(4,5-dimethylthiazol-2-yl)-2,5-diphenyltetrazolium bromide) (Biomatik, Wilmington, DE) assays were performed by plating 7,500 cells/well in 10% FBS/DMEM in a 96-well plate, followed by incubation at 37° C. for 24 h. Cells were then treated with vehicle or increasing concentrations of tcyDTDO, dMtcyDTDO, or dFtcyDTDO and incubated at 37° C. for 72 h. Cell media was subsequently removed from each well and MTT assays were initiated by incubating cells with 0.5 mg/ml MTT in PBS for 3 h, followed by removal of the MTT solution and addition of 100 □L of DMSO to each well for 30 min. Absorbance of the MTT formazan product was subsequently measured at 570 and 690 (reference) nm in a plate reader.

### In Vivo Tumor Studies and Histochemical Analysis

Breast cancer liver metastasis was initiated in NOD-SCID-gamma (NSG) mice obtained from Jackson Laboratories (Bar Harbor, ME) by injecting 1×10⁶ cancer cells into the #4 mammary fat pads as described previously (Wang M, et al. Cell Death Discovery 2019; 5: 153). Mice bearing HCI-001/LVM2 breast tumors were treated once daily for four consecutive days with 20 mg/kg tcyDTDO by oral gavage and livers were collected. Animals bearing HER2+ BT474 tumors were treated with the vehicle or 20 mg/kg dMtcyDTDO or dFtcyDTDO by intraperitoneal injection for four consecutive days. Tissue samples fixed in 4% paraformaldehyde/PBS and paraffin-embedded were sectioned and stained with hematoxylin and eosin (H&E) by the University of Florida Molecular Pathology Core (https://molecular.pathology.ufl.edu/).

### Streptavidin-Agarose Pulldowns

Cells were treated with Biotin-PyrDTDO with or without the pan-Caspase inhibitor Q-VD-OPH (MedChemExpress, Monmouth Junction, NJ) for 16 h in 10% FBS-DMEM, followed by the addition of 25 mM N-ethylmaleimide (NEM) (Thermo Scientific, Rockford, IL) for 20 min at 37° C. Cells were subsequently scraped directly into the media, washed twice with ice-cold PBS, and pelleted by centrifugation. Cells were extracted in buffer (20 mM HEPES (pH 7.4), 1 mM EDTA, 1mM EGTA, 5% Glycerol, 1 nM Microcystin, 1 mM Na₃VO₄, and 40 mM Na₂H₂P₂O₇) containing 1% Triton X-100. Samples were sonicated, followed by centrifugation for 20 min at 4° C. The supernatant was pre-cleared with Sepharose CL-6B (Amersham Biosciences AP, Uppsala, Sweden) for 2 h at 4° C. Pre-cleared samples were then incubated with Streptavidin (SA)-Agarose (Thermo Scientific, Rockford IL) and mixed on a Nutator for 24 h at 4° C. SA-Agarose was washed three times with extraction buffer containing 0.1% Triton X-100, followed by the addition of 2×-SDS Sample buffer containing 2 mM biotin. Samples were analyzed by immunoblot, silver stain, or probing with Streptavidin-Alkaline Phosphatase.

### Disulfide Bond-Mediated Oligomerization

To assess protein disulfide bond-mediated oligomerization, cells were treated with DDAs for 24 h in 10% FBS-DMEM. Cells were subsequently scraped directly into the media and pelleted by centrifugation. Cells were extracted directly into boiling 2×-SDS-Sample Buffer containing 100 mM NEM and boiled for 10 min. Samples were sonicated, followed by centrifugation for 20 min at room temperature. Samples were subsequently analyzed by SDS-PAGE and immunoblot.

### Surface Biotinylation

Cell surface proteins were labeled by incubation of cells with 1.6 mM Sulfo-NHS-SS-Biotin (Thermo Fisher Scientific, Waltham, MA) or 1.6 mM Biotin-dPEG₃-Maleimide (MilliporeSigma, St. Louis, MO) in Phosphate-Buffered Saline (PBS), pH 8.0 for 30 minutes at 4° C. The cells were subsequently washed three times with PBS, and proteins were extracted in a buffer containing 1% Triton X-100 extraction buffer in the absence of reducing agents. Cell extracts were then incubated with Streptavidin-agarose beads (Thermo Fisher Scientific, Waltham, MA) for 2.5 h at 4° C. Following centrifugation, the supernatant containing non-biotinylated proteins (flow-through) was retained and boiled in 2×-SDS-Sample Buffer containing 1% 2-mercaptoethanol. Streptavidin-agarose beads bound to biotinylated proteins were washed four times with 0.1% Triton X-100 extraction buffer, followed by boiling in 2×-SDS-Sample Buffer containing 1% 2-mercaptoethanol. Samples were then analyzed by immunoblot or blotting with Streptavidin-Alkaline Phosphatase.

### Construction of AGR2 Vectors

Total RNA from BT474 cells was extracted with TRIzol Reagent (Invitrogen, Waltham, MA) according to the manufacturer's protocol. Total cellular RNA was used to synthesize first-strand cDNA by using the PCR conditions listed: 25° C. for 10 min, 42° C. for 30 min, and 95° C. for 5 min. PCR was subsequently performed using the following AGR2 primers: 5′-TTTTGGATCCCACCATGGAGAAAATTCCAGTG-3′ and 5′-TTTTTACGTATTACAATTCAGTCTTCAG-3′. The PCR fragment encoding AGR2 was subsequently digested with BamHI and SnaBI and cloned into the 5′ BamHI and 3′ SnaBI sites of pMXs-IRES-Blasticidin. The AGR2/pMXs-IRES-Blasticidin vector was used as a template for PCR reactions using the following AGR2 primers to amplify the DNA sequence downstream of the AGR2 signal sequence and to add 5′-BamHI and 3′-SalI sites: 5′-TTTTGGATCCCAGAGATACCACAGTCAAAC-3′ and 5′-TTTTGTCGACTTACAATTCAGTCTTC-3′. The PCR fragment was digested with BamHI and SalI and cloned into the 5′ BamHI and 3′ SalI sites of pET-45b(+) (Novagen Burlington, MA). QuikChange mutagenesis was performed to produce the C81S mutation of AGR2 using the follow primers: 5′- CATCACTTGGATGAGTCCCCACACAGTCAAGC-3′ and 5′-GCTTGACTGTGTGGGGACTCATCCAAGTGATG-3′. The C81S mutation of AGR2 was verified by sequencing.

### His₆-AGR2 TALON Purification

Competent BL21(DE3) cells were transformed with either the AGR2/pET-45b(+) or AGR2 C81S/pET-45b(+) vectors. Protein expression was induced by the addition of 50 □M Isopropyl β-D-1-thiogalactopyranoside (Fisher Scientific, Fair Lawn, NJ) to bacterial cell cultures for 2.5 hours and shaking at 37° C. Bacterial cells were subsequently pelleted and dissolved in Mouse Tonicity Phosphate Buffered Saline (MTPBS) containing 1% Triton X-100 and sonicated for 20 seconds on ice, followed by centrifugation for 10 min at 4° C. to pellet cell debris. The supernatant was transferred to a new tube, followed by the addition of TALON Resin (Clontech, Mountain View, CA) and mixing on a Nutator for 30 min at 4° C. The TALON Resin was pelleted by centrifugation and washed twice with MTPBS and twice with MTPBS containing 30 mM Imidazole. Protein bound to the TALON Resin was eluted with 400 mM Imidazole and precipitated with 60% saturated ammonium sulfate. The precipitated protein was pelleted by centrifugation, dissolved in MTPBS containing 1 mM DTT and 1 mM EDTA, and subsequently dialyzed against a solution of MTPBS containing 10 μM DTT and 30% Glycerol.

### DDA-AGR2 Binding Experiments

Purified His₆-AGR2 or His₆-AGR2 C81S, as prepared above, or His₆-AGR2 (PRO-580) or His₆-AGR3 (PRO-242) obtained from ProSpec (New Brunswick NJ) were incubated with or without varying concentrations of the unlabeled DDA competitors, tcyDTDO or dMtcyDTDO, and 30 μM Biotin-GlyPyrDTDO or Biotin-PyrDTDO in buffer (20 mM HEPES (pH 7.4), 1 mM EDTA, 1 mM EGTA, 5% Glycerol, 1 nM Microcystin, 1 mM Na₃VO₄, and 40 mM Na₂H₂P₂O₇) containing 1% Triton-X 100 for 30 min at 37° C. Preliminary studies showed that Biotin-GlyPyrDTDO or Biotin-PyrDTDO exhibited indistinguishable labeling of proteins in intact cells and purified proteins in vitro. Therefore, the two probes were used interchangeably. Reactions were stopped by boiling the samples in 2×-SDS-Sample Buffer containing 100 mM NEM for 10 min. Biotin-DDA labeling of protein was verified by blotting with Streptavidin-Alkaline Phosphatase (Rockland Immunochemicals, Inc., Limerick, PA). ERp44 (PRO-547) and PDIA1 (ENZ-262) were obtained from ProSpec and used in experiments examining the binding of these proteins to Biotin-PyrDTDO.

### Sample Preparation for Mass Spectrometry

Total proteins were solubilized in 50 mM NH₄HCO₃ pH 8.5, and Trypsin (Promega, Fitchburg, WI) was added for digestion (w/w for enzyme:sample=1:100) overnight at 37° C. The digested peptides were desalted using micro ZipTip mini-reverse phase (Millipore), and then lyophilized to dryness.

### Nano-LC-MS

Peptides derived from the protein samples were suspended in 0.1% formic acid for mass spectrometric analysis. The bottom-up proteomics data acquisition was performed on an EASY-nLC 1200 ultra-performance liquid chromatography system connected to an Orbitrap Fusion Tribrid instrument equipped with a nano-electrospray source (Thermo Scientific, San Jose, CA). The peptide samples were loaded onto a C18 trapping column (75 μm i.d.×2 cm, Acclaim PepMap® 100 particles with 3 μm size and 100 Å pores) and eluted onto a C18 analytical column (75 μm i.d.×25 cm, 2 μm particles with 100 Å pore size). The flow rate was set at 300 nL/minute with solvent A (0.1% formic acid in water) and solvent B (0.1% formic acid and 99.9% acetonitrile) as the mobile phases. Separation was conducted using the following gradient: 2% of B over 0-0.5 min; 2-35% of B over 0.5-45 min, 35-98% of B over 45-46 min, and isocratic at 98% of B over 46-59 min, and then from 98-2% of B from 59-60 min. The equilibration time at 2% B is 15 min.

An Orbitrap with a resolution of 120,000 at m/z 400 was used for a full MS1 scan (m/z 350-2000). The automatic gain control (AGC) target is 2e5 with 50 ms as the maximum injection time. Monoisotopic precursor selection (MIPS) was enforced to filter for peptides. Peptides bearing +2-6 charges were selected with an intensity threshold of 1e4. Dynamic exclusion of 15 s was used to prevent resampling of high abundance peptides. Top speed method was used for data dependent acquisition within a cycle of 3 s. The MS/MS was carried out in the ion trap, with a quadrupole isolation window of 1.3 Da. Fragmentation of the selected peptides by collision-induced dissociation (CID) was done at 35% of normalized collision energy with 10 ms activation time. The MS2 spectra were detected in the linear ion trap with the AGC target as 1e4 and the maximum injection time as 35 ms.

Database searching was performed on all MS/MS samples using Mascot (Matrix Science, London, UK; version 2.4.1), with a fragment ion mass tolerance of 0.50 Da, a parent ion tolerance of 10.0 PPM. Mascot was set up to search the IPI human database assuming the digestion enzyme trypsin. MS/MS based peptide and protein identifications were made with Scaffold (version Scaffold_4.2.1, Proteome Software Inc., Portland, OR). Peptide Prophet algorithm (Keller A, et al. Anal Chem 2002; 74: 5383-5392) with Scaffold delta-mass correction was utilized to accept peptide identification at greater than 95.0% probability. Protein identifications were accepted if they contained a minimum of 1 identified peptide and could be established at greater than 95.0% probability. The Protein Prophet algorithm (Nesvizhskii A I, et al. Anal Chem 2003; 75: 4646-4658) was utilized to assign protein probabilities. Proteins were grouped into clusters if they shared significant peptide evidence.

### Statistics

Student's t-test was used for comparisons in both in vitro and in vivo experiments. All P values are two-tailed, and both P values and statistical tests are mentioned in either figures or legends. Points plotted on line graphs are the average of six or more technical replicates and are representative of three or more independent biological replicates. Error bars represent standard deviation of the mean. Immunoblot analyses are representative of at least three independent replicates with similar results.

### Chemical Synthesis of DDAs

Reagents and solvents were purchased from commercial sources and used without further purification unless otherwise specified. Anhydrous solvents were obtained using a commercial solvent drying system (using activated alumina for THF, diethyl ether, dichloromethane; molecular sieves for DMF) and transferred via syringe to flame-dried glassware that had been cooled under an argon atmosphere. ¹H and ¹³C NMR spectra were recorded using commercially-obtained deuterated solvents on Bruker-600 (¹H at 600 MHz; ¹³C at 151 MHz) and Varian Inova-500 (¹H at 500 MHz; ¹³C at 125 MHz) spectrometers. Chemical shifts (δ) are given in parts per million (ppm) relative to TMS and referenced to residual protonated solvent (CDCl₃: δ_{H} 7.26 ppm, δ_{C} 77.16 ppm; CD₃OD: δ_{H} 4.87 ppm, δ_{C} 49.00 ppm; DMSO-d₆: δ_{H} 2.50 ppm, δ_{C} 39.52 ppm; D₂O: δ_{H} 4.79 ppm). Coupling constants (J) are reported in Hz. Spin multiplicities are presented by the following symbols: s (singlet), bs (broad singlet), d (doublet), t (triplet), q (quartet), p (pentet), and m (multiplet). Electrospray ionization (ESI) high-resolution mass spectra (HRMS) were recorded on an ESI-TOF instrument, operating in positive mode as stated, with methanol as the carrier solvent otherwise as mentioned.

### Synthesis of (±)-BocPyrDTDO

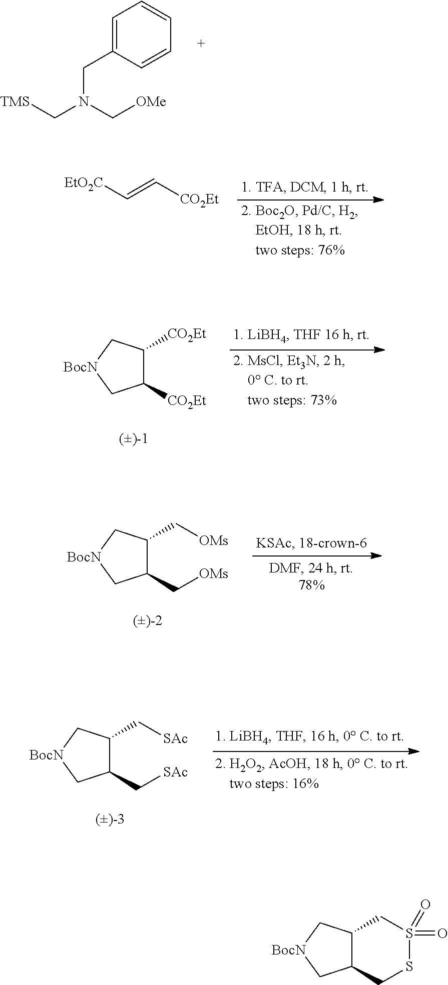

### 1-(tent-Butyl)-3,4-diethyl-(3S,4S)- and (3R,4R)-pyrrolidine-1,3,4-tricarboxylate, (±)-1

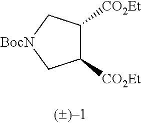 [SMILES: CCOC(=O)[C@H]1CN(C(=O)OC(C)(C)C)C[C@@H]1C(=O)OCC]

To a stirred solution of diethyl fumarate (3.8 mL, 23 mmol) in DCM (50 mL) at 0° C. was added N-benzyl-1-methoxy-N-((trimethylsilyl)methyl)methanamine (5.4 mL, 21 mmol) followed by dropwise addition of a solution of TFA in DCM (0.1 mL of TFA in 1 mL of DCM) over 10 min. After completion of addition, the cold bath was removed, and the reaction mixture was stirred at rt for 1 h. The reaction mixture was diluted with DCM (20 mL), washed with a saturated solution of sodium bicarbonate (20 mL), and the organic layer was dried over Na₂SO₄ and concentrated under reduced pressure. The residue was dissolved in ethyl acetate (25 mL) and to this was added Boc₂O (8.00 g, 36.6 mmol) followed by 10% palladium on carbon (1 g, wet). The resulting reaction mixture was subjected to catalytic hydrogenation using hydrogen gas for 18 h with stirring. The catalyst was filtered through a pad of celite, and the filtrate was concentrated. The residue was purified by flash silica gel column chromatography (0-25% EtOAc/hexanes) to provide the Boc-protected diester (±)-1 (5.1 g, 16 mmol, 76% yield) as a pale-yellow oil. ¹H NMR (CDCl₃, 500 MHz): δ4.16 (q, J=7.1 Hz, 4H), 3.78-3.69 (m, 2H), 3.57-3.44 (m, 2H), 3.42-3.29 (m, 2H), 1.45 (s, 9H), 1.26 (t, J=7.1 Hz, 6H). ¹³C NMR (CDCl₃, 125 MHz): δ171.81, 154.02, 80.02, 61.44, 48.12, 48.02, 46.12, 45.39, 28.54, 14.23.

### tert-Butyl (3S,4S)- and (3R,4R)-3,4-bis(((methylsulfonyl)oxy)methyl)pyrrolidine-1-carboxylate, (±)-2

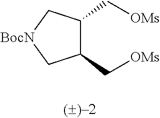 [SMILES: CC(C)(C)OC(=O)N1C[C@H](COS(C)(=O)=O)[C@@H](COS(C)(=O)=O)C1]

To an ice-cooled solution of the diester (±)-1 (5.0 g, 16 mmol) in anhydrous THF (135 mL) was added dropwise a solution of LiBH₄ in THF (16 mL of a 2.0 M LiBH₄ solution in THF diluted with an additional 20 mL of anhydrous THF). The reaction mixture was stirred overnight at rt and quenched with an aqueous 2 M NaOH solution. Ether was added, the layers were separated, and the aqueous phase was back extracted twice with ether. The combined organic extracts were dried over Na₂SO₄, filtered, and concentrated to give the diol which was used without further purification. To a stirred solution of the diol in DCM (110 mL) cooled to 0° C. was added triethylamine (11 mL, 80 mmol) followed by methanesulfonyl chloride (3.1 mL, 40 mmol). The reaction mixture was stirred at 0° C. for 15 min, then at rt for 1.5 h. The reaction mixture was poured into water (76 mL) and the phases were separated. The aqueous phase was extracted with DCM (2×76 mL). The combined organics were dried (MgSO₄), filtered, and concentrated. The residue was purified by flash chromatography (0-100% EtOAc/DCM) to afford the desired dimesylate (±)-2 (4.5 g, 12 mmol, 73% yield) as a white solid. ¹H NMR (CDCl₃, 500 MHz): δ4.25 (m, 4H), 3.65 (m, 2H), 3.24 (m, 2H), 3.05 (s, 6H), 2.53 (m, 2H), 1.46 (s, 9H). ¹³C NMR (CDCl₃, 125 MHz): δ154.24, 80.23, 68.86, 47.95, 47.46, 40.52, 39.42, 37.70, 28.58.

### tert-Butyl-(3S,4S)- and (3R,4R)-3,4-bis((acetylthio)methyl)pyrrolidine-1-carboxylate, (±)-3

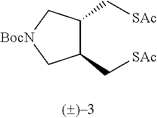

A mixture of the dimesylate (±)-2 (7.8 g, 20 mmol), KSAc (4.6 g, 40 mmol), and 18-crown-6 (1.3 g, 5.0 mmol) in anhydrous DMF (392 mL) was stirred for 24 h at rt. After completion of the reaction, water (392 mL) was added and the mixture was extracted with EtOAc (3×392 mL). The combined organic phases were washed with water (392 mL) and brine (392 mL), dried (Na₂SO₄), concentrated, and purified by column chromatography (0-10% DCM/EtOAc) to afford the dithioacetate (±)-3 (5.4 g, 16 mmol, 78% yield) as a colorless oil. ¹H NMR (CDCl₃, 500 MHz): δ3.69-3.50 (m, 2H), 3.20-3.09 (m, 2H), 3.09-2.93 (m, 2H), 2.85 (dd, J=13.7, 7.3 Hz, 2H), 2.34 (s, 6H), 2.22-2.05 (m, 2H), 1.44 (s, 9H). ¹³C NMR (CDCl₃, 125 MHz): δ195.26, 154.37, 79.63, 50.64, 50.29, 43.13, 42.40, 30.74, 30.65, 28.60. HRMS (ESI-TOF): Calculated for [C₁₅H₂₅NO₄S₂+Na]⁺: 370.1117; found: 370.1104.

### tert-Butyl-(4aS,7aS)- and (4aR,7aR)-tetrahydro-1H-[1,2]dithiino[4,5-e]pyrrole-6(4H)-carboxylate 2,2-dioxide, (±)-BocPyrDTDO

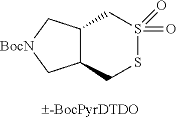 [SMILES: CC(C)(C)OC(=O)N1C[C@@H]2CS(=O)(=O)SC[C@H]2C1]

To an ice-cooled solution of the dithioacetate (±)-3 (5.42 g, 15.6 mmol) in anhydrous THF (132 mL) was added dropwise a solution of LiBH₄ in THF (7.8 mL of 4.0 M LiBH₄ in THF diluted with additional 27 mL of anhydrous THF). The reaction mixture was stirred overnight at rt and quenched with an aqueous 2 M NaOH solution. Ether (25 mL) was added, the layers were separated, and the aqueous phase was back extracted with ether (2×25 mL). The combined organic extracts were dried over Na₂SO₄, filtered, and concentrated to give the crude dithiol (3.81 g, 14.5 mmol, 93% yield), which was used without further purification for the next step. A solution of the crude dithiol (1.91 g, 7.23 mmol) in AcOH (9.0 mL) was cooled in an ice bath and a solution of H₂O₂ in water/AcOH (2.59 mL of 30% H₂O₂ in water diluted with 3.0 mL of AcOH) was added slowly such that the reaction temperature did not rise above 35° C. After stirring for 18 h, the solvent was removed under vacuum, and the residue was diluted with water (15 mL), neutralized with NaHCO₃, and extracted with EtOAc (3×50 mL). The organic extract was dried with Na₂SO₄, filtered, and concentrated under vacuum. The crude material was purified by column chromatography (0-100% EtOAc/DCM) to afford the desired (±)-BocPyrDTDO (0.66 g, 2.25 mmol, 16% yield). ¹H NMR (CDCl₃, 500 MHz): δ3.75 (m, 1H), 3.67 (m, 1H), 3.52 (m, 1H), 3.41 (t, J=12.6 Hz, 1H), 3.33 (t, J=12.6, 1H), 3.14 (m,1H), 3.07 (t, J=10.8 Hz, 1H), 3.01 (t, J=10.8 Hz, 1H), 2.8-2.67 (m, 1H), 2.40-2.28 (m, 1H), 1.45 (s, 9H). ¹³C NMR (CDCl₃, 125 MHz): δ154.09, 80.27, 62.62, 62.50, 50.16, 49.76, 49.63, 49.19, 43.14, 43.01, 42.44, 42.36, 35.43, 35.30, 28.55. HRMS (ESI-TOF): Calculated for [C₁₁H₁₉NO₄S₂+Na]⁺: 316.0648; found: 316.0646. Slow s-cis/s-trans interconversion with respect to the N-acylpyrrolidine ring produces two conformational diastereomers and additional NMR signals at room temperature. The interconversion is fast on the NMR timescale at 75° C. in DMSO-d₆: ¹H NMR (DMSO-d₆, 500 MHz) at 75° C.: δ3.80 (dd, J=13.1, 2.8 Hz, 1H), 3.63-3.53 (m, 3H), 3.43 (dd, J=13.5, 2.9 Hz, 1H), 3.32 (dd, J=13.5, 11.4 Hz, 1H), 3.03 (t, J=10.6 Hz, 1H), 2.97 (t, J=10.6 Hz, 1H), 2.65-2.53 (m, 1H), 2.41-2.31 (m, 1H), 1.43 (s, 9H). ¹³C NMR (DMSO-d₆, 125 MHz) at 75° C.: δ153.87, 79.12, 62.83, 50.04, 49.58, 42.78, 42.01, 35.55, 28.70. High temperature 1D and 2D NMR spectra for this compound are included.

### Synthesis of BioGlyPyrDTDO (BGPD)

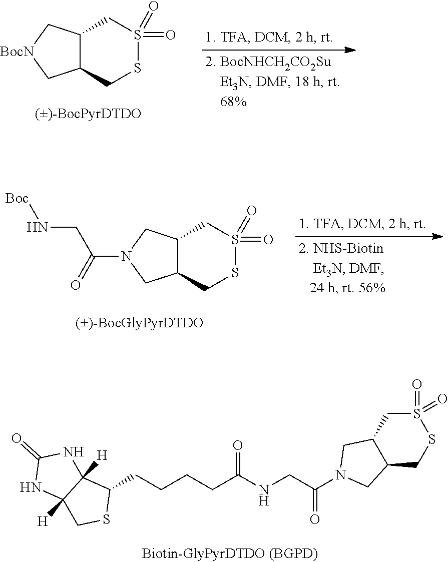 [SMILES: CC(C)(C)OC(=O)N1C[C@@H]2CS(=O)(=O)SC[C@H]2C1 ; CC(C)(C)OC(=O)NCC(=O)N1C[C@@H]2CS(=O)(=O)SC[C@H]2C1 ; O=C(CCCC[C@@H]1SC[C@@H]2NC(=O)N[C@@H]21)NCC(=O)N1C[C@@H]2CS(=O)(=O)SC[C@H]2C1]

### tert-Butyl-(2-((4aS,7aS)- and (4aR,7aR)-2,2-dioxidotetrahydro-1H-[1,2]dithiino[4,5-c]pyrrol-6(4H)-yl)-2-oxoethyl)carbamate, (±)-BocGlyPyrDTDO

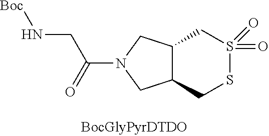 [SMILES: CC(C)(C)OC(=O)NCC(=O)N1C[C@@H]2CS(=O)(=O)SC[C@H]2C1]

A solution of (±)-BocPyrDTDO (0.10 g, 0.34 mmol) was stirred in TFA:DCM (3.4 mL, 1:1) for 2 h, at rt. The reaction mixture was then concentrated under vacuum to provide the deprotected ammonium trifluoroacetate salt HPyrDTDO.TFA as a brown solid, which was used in the next step without further purification. ¹H NMR (DMSO-d₆, 500 MHz): δ9.25 (bs, 2H), 3.91 (dd, J=13.0, 2.9 Hz, 1H), 3.68 (t, J=12.6 Hz, 1H), 3.53-3.42 (m, 3H), 3.33 (dd, J=13.6, 11.5 Hz, 1H), 2.95 (tq, J=13.4, 6.6 Hz, 2H), 2.62-2.53 (m, 1H), 2.40-2.30 (m, 1H). ¹³C NMR (DMSO-d₆, 125 MHz): δ60.97, 47.55, 47.30, 41.98, 40.64, 33.97.

To the solution of the resulting solid in DMF (4.8 mL) was added Et₃N (0.10 mL, 0.68 mmol) and 2,5-dioxopyrrolidin-1-yl (tert-butoxycarbonyl)glycinate (0.09 g, 0.34 mmol), respectively, and the reaction mixture was stirred at rt for 24 h. After this time the solvent was evaporated, and the crude product was purified by flash chromatography (0-1% MeOH/DCM) to afford (±)-BocGlyPyrDTDO (0.08 g, 0.23 mmol 68%) as a viscous white solid. ¹H NMR (CDCl₃, 500 MHz): δ5.38 (s, 1H), 4.01-3.95 (m, 1H), 3.93-3.79 (m, 2H), 3.74 (dt, J=16.0, 8.5 Hz, 1H), 3.58 (t, J=13.9 Hz, 1H), 3.48-3.34 (m, 2H), 3.26-3.04 (m, 3H), 2.9-2.7 (m, 1H), 2.53-2.29 (m, 1H), 1.44 (s, 9H). ¹³C NMR (CDCl₃, 125 MHz): δ167.38, 155.92, 80.06, 62.42, 62.14, 49.89, 49.38, 48.94, 43.39, 43.14, 42.96, 42.88, 41.72, 41.60, 35.18, 34.95, 28.46. HRMS (ESI-TOF): Calculated for [C₁₃H₂₂N₂O₅S₂+Na]⁺: 373.0862; found: 373.0863. Slow s-cis/s-trans interconversion with respect to the N-acylpyrrolidine ring produces two diastereomers and additional NMR signals (e.g., PyrDTDO ring peak doubling) at room and even elevated (in DMSO-d₆) temperature.

### N-(2-((4aS,7aS)- and (4aR,7aR)-2,2-Dioxidotetrahydro-1H-[1,2]dithiino[4,5-c]pyrrol-6(4H)-yl)-2-oxoethyl)-5-((3aS,4S,6aR)-2-oxohexahydro-1H-thieno[3,4-d]imidazol-4-yl)pentanamide, Biotin-G1yPyrDTDO (BGPD)

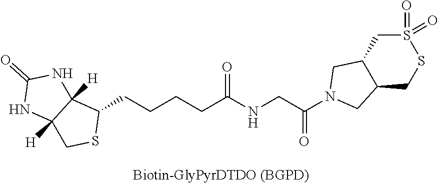 [SMILES: O=C(CCCC[C@@H]1SC[C@@H]2NC(=O)N[C@@H]21)NCC(=O)N1C[C@@H]2CS(=O)(=O)SC[C@H]2C1]

A solution of (±)-BocGlyPyrDTDO (0.08 g, 0.23 mmol) was stirred in TFA:DCM (6 mL, 1:2) for 2 h at rt. After this time the reaction was concentrated under vacuum to provide the deprotected ammonium trifluoroacetate salt as a brown oily solid, which was used in the next step without further purification. To a solution of the resulting solid and (+)-biotin N-succinimidyl ester (0.08 g, 0.23 mmol) in DMF was added Et₃N (0.06 mL, 0.46 mmol) and the reaction mixture was stirred at rt for 24 h. Then the solvent was evaporated and the crude product was purified by flash chromatography (0-10% MeOH/DCM) to afford Biotin-G1yPyrDTDO (0.06 g, 0.13 mmol, 56%) as a white solid. ¹H NMR (DMSO-d₆, 500 MHz) δ7.94 (s, 1H), 6.41 (s, 1H), 6.35 (s, 1H), 4.33-4.27 (m, 1H), 4.14-4.11 (m, 1H), 3.90-3.68 (m, 5H), 3.64 (td, J=12.9, 2.0 Hz, 1H), 3.46 (ddd, J=13.5, 6.4, 2.8 Hz, 1H), 3.35-3.30 (m, 1H), 3.24-3.17 (m, 1H), 3.11-3.05 (m, 1H), 3.02-2.91 (m, 1H), 2.82 (dd, J=12.4, 5.1 Hz, 1H), 2.69-2.47 (m, 2H), 2.46-2.25 (m, 1H), 2.14 (t, J=7.0 Hz, 2H), 1.65-1.58 (m, 1H), 1.55-1.42 (m, 3H), 1.39-1.25 (m, 2H). ¹³C NMR (DMSO-d₆, 125 MHz): δ172.31, 166.93, 166.86, 162.68, 61.79, 61.65, 61.00, 59.18, 55.41, 49.25, 48.76, 48.31, 42.72, 41.67, 41.05, 40.87, 40.78, 40.11, 34.94, 34.85, 34.80, 28.14, 28.02, 25.23. HRMS (ESI-TOF): Calculated for [C₁₈H₂₈N₄O₅S₃+H]⁺: 477.1295; found: 477.1287. Slow s-cis/s-trans interconversion with respect to the N-acylpyrrolidine ring produces two diastereomers and additional NMR signals (e.g., PyrDTDO ring peak doubling).

### Synthesis of Biotin-PyrDTDO (BPD)

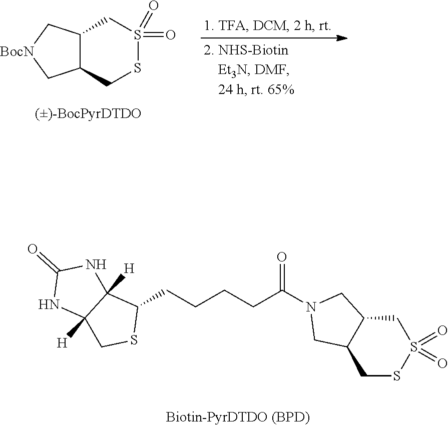 [SMILES: CC(C)(C)OC(=O)N1C[C@@H]2CS(=O)(=O)SC[C@H]2C1 ; O=C1N[C@H]2[C@H](CS[C@H]2CCCCC(=O)N2C[C@@H]3CS(=O)(=O)SC[C@H]3C2)N1]

### (3aS,4S,6aR)-4-(5-((4aR,7aR) and (4aS,7aS)-2,2-dioxidotetrahydro-1H-[1,2]dithiino[4,5-c]pyrrol-6(4H)-yl)-5-oxopentyptetrahydro-1H-thieno[3,4-d]imidazol-2(3H)-one, Biotin-PyrDTDO (BPD)

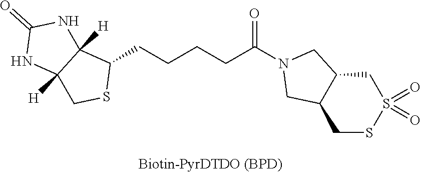 [SMILES: O=C1N[C@H]2[C@H](CS[C@H]2CCCCC(=O)N2C[C@@H]3CS(=O)(=O)SC[C@H]3C2)N1]

A solution of (±)-BocPyrDTDO (0.20 g, 0.68 mmol) was stirred in TFA:DCM (6 mL, 1:1) for 2 h at rt, monitoring by TLC. After reaction completion, the mixture was concentrated under vacuum to provide the deprotected ammonium trifluoroacetate salt as a brown solid, which was used in the next step without further purification. To a solution of the resulting solid and (+)-biotin N-succinimidyl ester in DMF was added Et₃N (0.20 mL, 1.40 mmol) and the reaction mixture was stirred at rt for 24 h. Then the solvent was evaporated and the crude product was purified by flash chromatography (0-10% MeOH/DCM) to afford Biotin-PyrDTDO (0.19 g, 0.45 mmol, 65%) as a white solid. ¹H NMR (DMSO-d₆, 600 MHz) δ6.44 (s, 1H), 6.36 (s, 1H), 4.31 (dd, J=7.8, 5.0 Hz, 1H), 4.14 (ddd, J=7.1, 4.5, 1.8 Hz, 1H), 3.88-3.82 (m, 1H), 3.76-3.66 (m, 3H), 3.65-3.60 (m, 1H), 3.49-3.40 (m, 1H), 3.28-3.35 (m, 1H), 3.23-3.13 (m, 1H), 3.13-3.07 (m, 1H), 2.99-2.87 (m, 1H), 2.83 (dd, J=12.4, 5.1 Hz, 1H), 2.58 (d, J=12.4 Hz, 1H), 2.67-2.47 (m, 1H), 2.45-2.28 (m, 1H), 2.26-2.13 (m, 2H), 1.68-1.57 (m, 1H), 1.56-1.42 (m, 3H), 1.40-1.27 (m, 2H). ¹³C NMR (DMSO-d₆, 151 MHz): δ170.42, 170.35, 162.71, 61.89, 61.69, 61.05, 59.18, 55.45, 49.67, 49.22, 49.02, 48.52, 42.70, 41.71, 41.23, 40.34, 39.87, 35.06, 34.86, 33.18, 33.11, 28.29, 28.11, 24.27. HRMS (ESI-TOF): Calculated for [C₁₆H₂₅N₃O₄S₃+Na]⁺: 442.0899; found: 442.0899. Slow s-cis/s-trans interconversion with respect to the N-acylpyrrolidine ring produces two diastereomers and additional NMR signals (e.g., PyrDTDO ring peak doubling).

### Characterization of DDAs with Enhanced Potency

DDAs, including RBF3, DTDO, and tcyDTDO (FIG. 1A), suppress tumor growth through induction of apoptotic cancer cell death. The HCI-001/LVM2 model was obtained from the HCI-001 Patient-Derived Xenograft by two rounds of selection for liver metastasis. Mice bearing HCI-001/LVM2 breast tumors were treated once daily for four consecutive days with 20 mg/kg tcyDTDO by oral gavage and livers were collected. Liver metastases in the tcyDTDO treated animals exhibited central necrosis (FIG. 1B, arrows). Although central necrosis can occur in tumors due to an insufficient blood supply, necrosis is not observed in vehicle treated tumors, indicating that the tumor cell death is caused by tcyDTDO treatment.

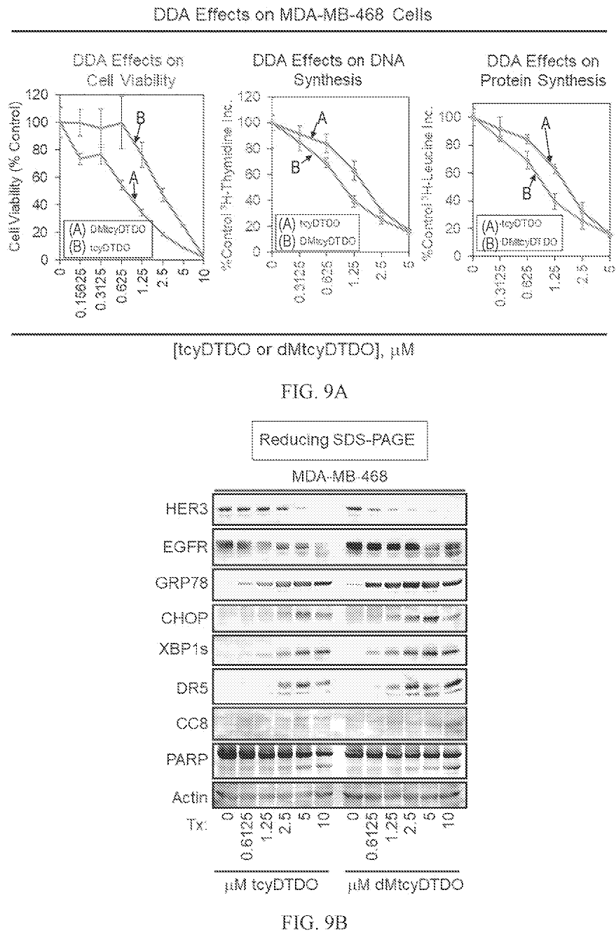

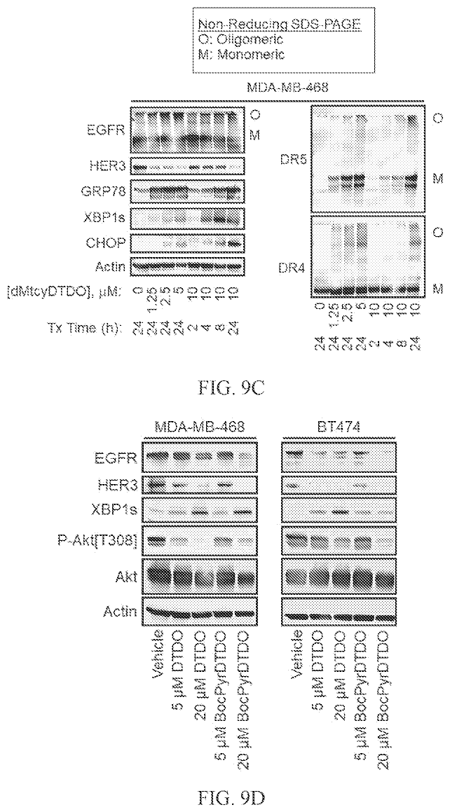

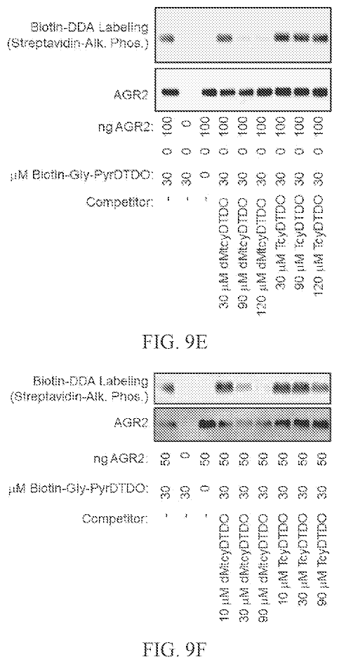

Modifications of the parent cyclic DDA, DTDO, provide a more potent DDA, tcyDTDO, with improved pharmacodynamic and pharmacokinetic properties. With tcyDTDO in hand, the effect of substitutions on its cyclohexane ring was then explored to determine if its potency and drug-like properties could be further improved. These efforts led to the design and synthesis of DDAs including dimethoxy-tcyDTDO (dMtcyDTDO) and difluoro-tcyDTDO (dFtcyDTDO) (FIG. 1C). Fluorine's size and high electronegativity lead to strong C—F bonds that resist metabolic degradation making fluorine substitution an effective way to increase drug stability. For instance, it has been shown that replacing hydrogen with fluorine significantly alters the rate and extent of Cytochrome P450 metabolism. Both dMtcyDTDO and dFtcyDTDO exhibit approximately two-fold greater potency than tcyDTDO in cell viability assays (FIG. 1D). DMtcyDTDO also exhibited two-fold greater potency than tcyDTDO in DNA and protein synthesis assays (FIG. 9A). DDAs were previously shown to induce apoptotic cell death associated with ERS, disulfide bond-mediated oligomerization of Death Receptors 4 and 5 (DR4/5) and EGFR, and upregulation of DR5. Therefore, the effect of the novel DDAs on disulfide bonding of these proteins by immunoblot analysis under non-reducing conditions was examined. When applied to cancer cells at the same concentration, dMtcyDTDO and dFtcyDTDO induced more DR4/5 oligomerization, higher DR5 levels, and produced greater ERS (FIG. 1E, left panel). Induction of ERS by dFtcyDTDO and dMtcyDTDO, as indicated by GRP78 elevation, and disulfide-mediated oligomerization of DR4 and EGFR, was detected at 625 nM and oligomerization of DR5 was observed at 1.25 μM of each compound (FIG. 1E, right panel). Additional immunoblot analyses performed under reducing conditions revealed that dMtcyDTDO is more potent than tcyDTDO in the induction of HER₃ downregulation, DR5 upregulation, and ERS as indicated by elevated CHOP and XBP1s (FIG. 9B). Time-course and concentration-response analyses of these endpoints showed that dMtcyDTDO induced these responses as early as four hours after treatment (FIG. 9C).

Quantitation of dMtcyDTDO effects on the levels and state of oligomerization of DR4 and DR5 revealed that dMtcyDTDO increases DR4 oligomerization without affecting overall DR4 levels, while dMtcyDTDO increases the levels of both oligomeric and monomeric forms of DR5 (FIG. 1F). Tumor studies using the HER2+ BT474 xenograft model were performed to examine if dMtcyDTDO and dFtcyDTDO induce rapid breast cancer cell death as observed with tcyDTDO. Tumors from animals treated with the vehicle for four consecutive days did not show evidence of tumor necrosis (FIG. 1G). In contrast, tumors were not observed in mammary fat pads from dFtcyDTDO treated animals. Mammary glands from dMtcyDTDO treated animals exhibited tumors that had undergone coagulation necrosis, consistent with rapid and uniform death of the cancer cells.

### AGR2 as a DDA Binding Protein

To understand how DDAs kill breast tumors, knowledge of their direct protein targets is essential. The development of molecular probes proved important in identifying DDA biological targets and linking them to the observed DDA responses. Furthermore, knowledge of the DDA binding site enables rational design of optimized next-generation DDAs. For this purpose, activity-based protein profiling and standard affinity pulldown techniques with biotin as the affinity label were used.

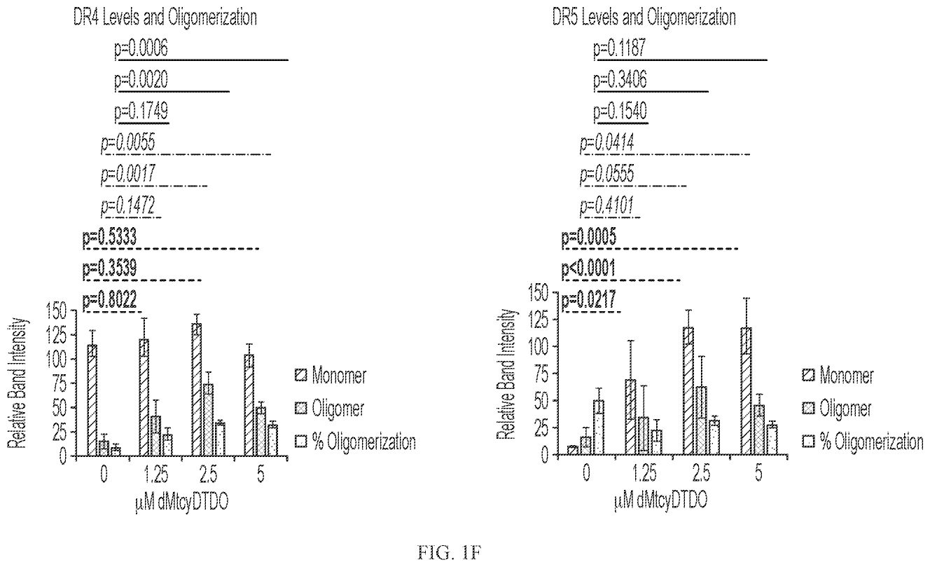

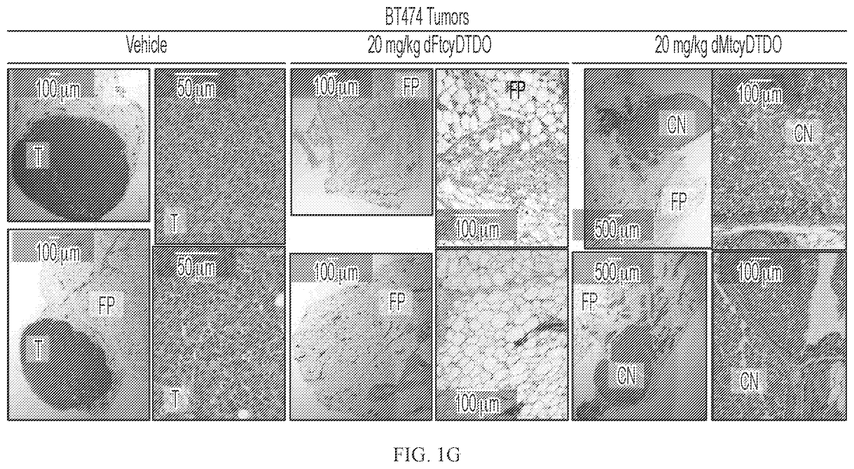

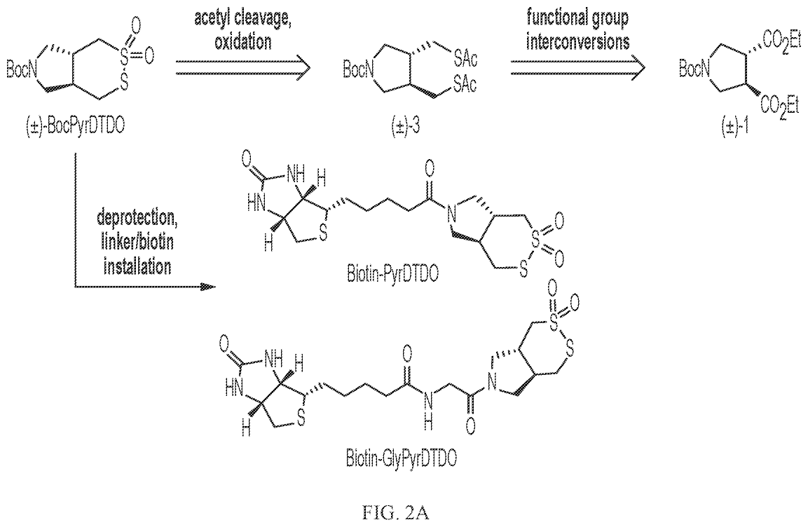

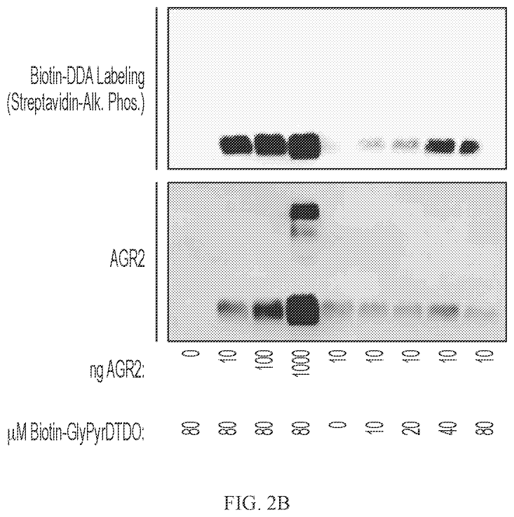

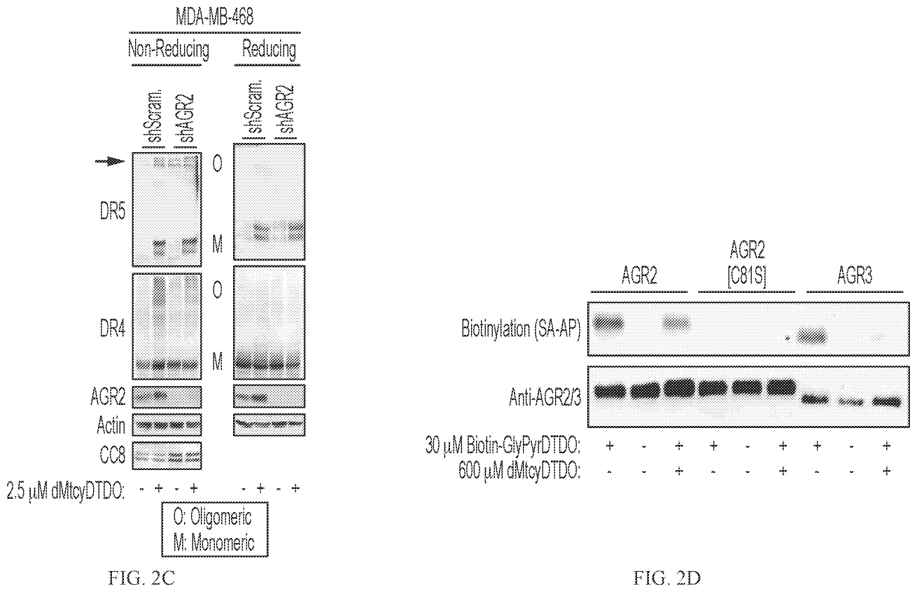

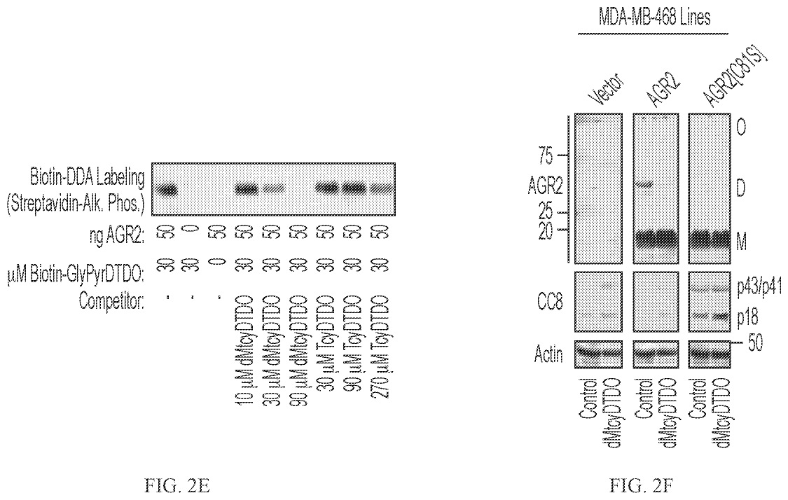

To generate DDA probes, a biologically active cyclic DDA with a functional group that enables installation of a biotin tag was employed. This was achieved by fusing a Boc-protected pyrrolidine ring at the 4,5-positions of the parent DTDO compound to generate (±)-BocPyrDTDO (FIG. 2A), which is biologically active and showed a potency similar to DTDO (FIG. 9D). The synthesis of (±)-BocPyrDTDO was accomplished by cleaving the acetyl groups of dithioacetate (±)-3 followed by oxidation to furnish the 1,2-dithiane 1,1-dioxide ring (FIG. 2A). Intermediate (±)-3 can be obtained through a few functional group interconversion steps from diester (±)-1 which itself could be obtained according to literature procedures. The amino group on the pyrrolidine ring, after Boc group deprotection, enables installation of the linker and biotin tag as described in full detail in the Supplementary Information. Biotinylated DDA analogs, including Biotin-PyrDTDO (BPD) and Biotin-GlyPyrDTDO (BGPD), were generated in this fashion and used as affinity tags to identify DDA targets (FIG. 2A).

The protein disulfide isomerase (PDI) family member AGR2 was previously implicated in disulfide bonding of EGFR, and DDAs alter EGFR disulfide bonding (FIG. 1E, right panel). AGR2 was hypothesized to be a target of DDAs and examined to determine whether biotinylated DDAs bind covalently with recombinant AGR2. Biotin-GlyPyrDTDO labeled AGR2 in a concentration-dependent manner (FIG. 2B). In addition to EGFR, DR4 or DR5 were hypothesized as possible AGR2 client proteins. Stable knockdown of AGR2 in breast cancer cells increased disulfide-mediated DR5 oligomerization and increased Caspase 8 cleavage/activation (FIG. 2C, CC8). AGR2 forms mixed disulfide bonds to its client proteins with Cys81 in its thioredoxin-like repeat, and Cys81 is the only Cys residue present in AGR2. Therefore, mutating Cys81 to Serine prevented DDA labeling was examined. The C81S AGR2 mutant was not labeled by Biotin-GlyPyrDTDO (FIG. 2D). AGR3 is the PDI paralog most closely related to AGR2 and it was also labeled by Biotin-GlyPyrDTDO. Addition of a molar excess of dMtcyDTDO to the labeling reactions partially prevented Biotin-GlyPyrDTDO binding to AGR2 and AGR3 (FIG. 2D), suggesting competition for binding to the same Cys residue(s). A comparison of the ability of dMtcyDTDO and tcyDTDO to block Biotin-GlyPyrDTDO binding to AGR2 indicated that dMtcyDTDO was a more effective competitor, suggesting that competition in this binding assay reflects DDA potency (FIG. 2E, 9E-F). AGR2 is expressed at low levels in MDA-MB-468 cells, and disulfide-mediated AGR2 complexes with client proteins were not detected in either the presence or absence of dMtcyDTDO treatment (FIG. 2F). Enforced AGR2 expression revealed formation of dimeric AGR2 that was not observed with the C81S AGR2 active site mutant. Treatment of the cells with dMtcyDTDO blocked dimer formation by wild type AGR2, consistent with blockade of C81S by dMtcyDTDO binding.

### Identification of PDIA1 and ERp44 as DDA Target Proteins

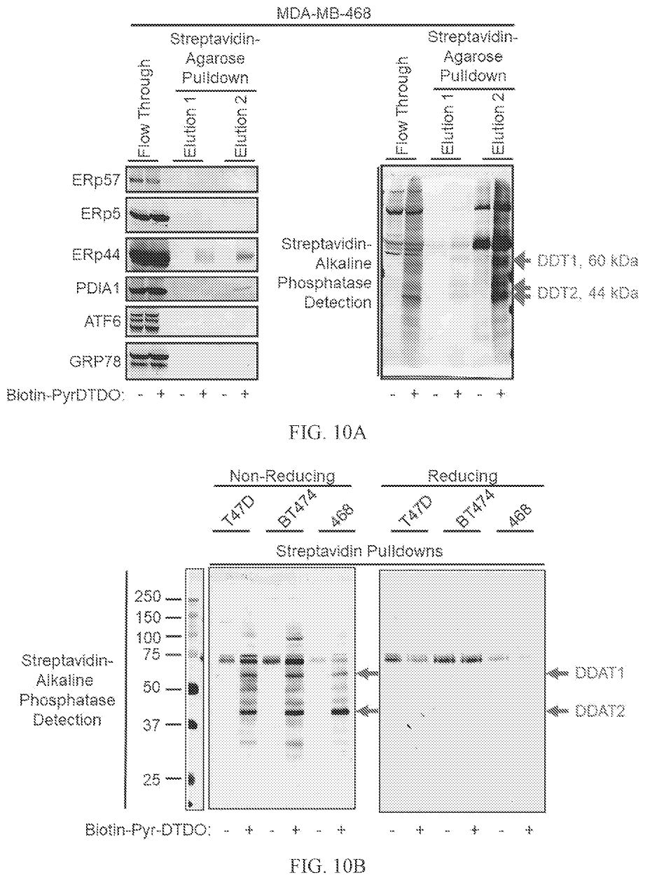

A panel of three breast cancer cell lines was treated with Biotin-PyrDTDO to label DDA target proteins in intact cells. Two biotinylated proteins that were observed exclusively in the context of cell labeling with Biotin-PyrDTDO were termed DDA target proteins 1 and 2 (DDAT1/2). DDAT1/2 migrated at 60 kDa and 44 kDa, respectively, and were observed in three different breast cancer lines in crude cell extracts and in samples purified using Streptavidin-agarose (FIG. 3A). The Streptavidin-agarose purification was scaled up to generate DDAT1/2 in sufficient amounts for identification by mass spectrometry, and small amounts of this preparation and the corresponding crude extracts were analyzed by blotting with Streptavidin-Alkaline Phosphatase, and by silver staining (FIG. 3B). DDAT1 and DDAT2 from a Coomassie stained gel were excised and analyzed by trypsin digestion followed by mass spectrometry as described in Materials and Methods. DDAT1 and DDAT2 were identified as the PDI-family members ERp44 and PDIA1, respectively. In support of these assignments, ERp44, PDIA1, and AGR2 were isolated from the luminal A T47D and luminal B BT474 breast cancer cell lines treated with Biotin-PyrDTDO using Streptavidin-Agarose (FIG. 3C, left panel). In additional Biotin-PyrDTDO pulldown experiments, ERp44 and PDIA1 were detected, but the PDIs ERp57 and ERp5 were not (FIG. 10A). Endogenous proteins are biotinylated via amide linkages. Thus, DDAT1 and DDAT2 are distinguished from endogenous biotinylated proteins by disruption of Biotin-DDA/target protein linkages with the reducing agent 2-Mercaptoethanol (FIG. 10B). Immunoprecipitation with an AGR2 antibody showed that ERp44 and PDIA1 co-purify with AGR2 from cells irrespective of treatment with Biotin-PyrDTDO (FIG. 3C, left panel). ERp44 immunoprecipitates contained PDIA1, but AGR2 co-purification was not detected. Biotin-PyrDTDO/Streptavidin-Agarose also affinity purified ERp44 and PDIA1 from the MDA-MB-468 Triple-Negative breast cancer cell line (FIG. 3C, right panel). In vitro binding studies showed that Biotin-PyrDTDO labeled ERp44 and PDIA1, and in both cases, biotinylation was blocked by pre-incubation with a molar excess of dMtcyDTDO (FIG. 3D). If ERp44 and PDIA1 are cellular DDA targets, then DDA treatment may alter the pattern of ERp44 and PDIA1 disulfide bonding with their client proteins. Consistent with this, treatment of breast cancer cells with dMtcyDTDO reduces disulfide bonding of ERp44 and PDIA1 with multiple proteins (FIG. 3E, red arrows) with minimal effect on monomeric levels of ERp44 and PDIA1. PDIA1 is a validated target for anticancer drugs. However, ERp44 has not been investigated extensively as a therapeutic target for cancer, and small molecule ERp44 inhibitors have not been reported. Similarly, small molecule AGR2 inhibitors have not been reported. Therefore, efforts were focused on characterizing AGR2 and ERp44 as DDA target proteins.

### DDAs Block the ER Retention Function of ERp44

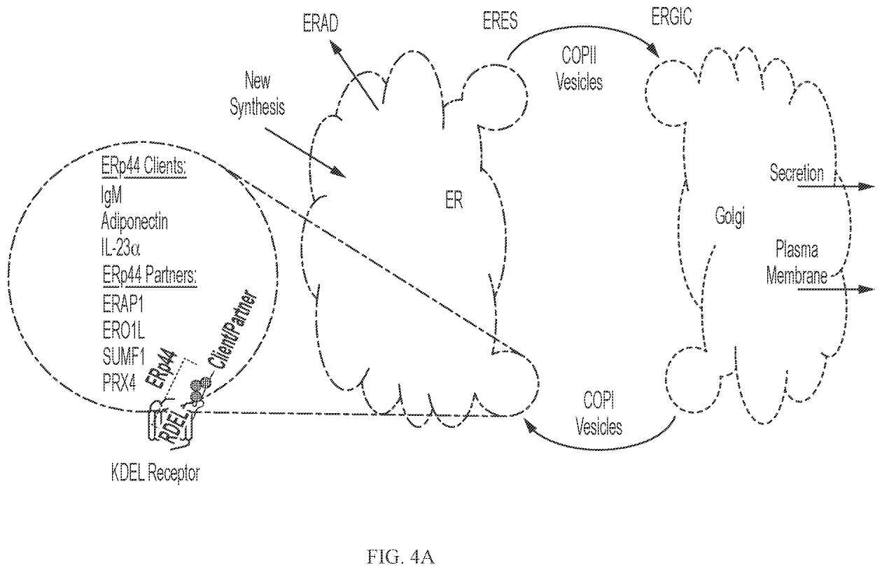

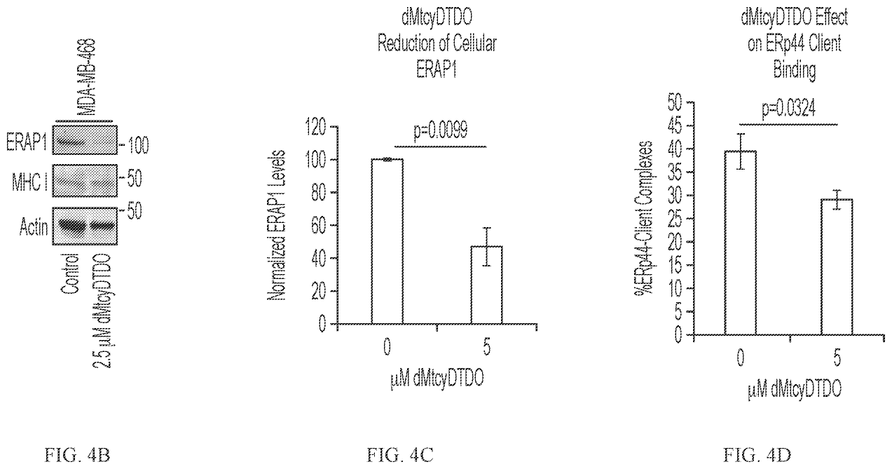

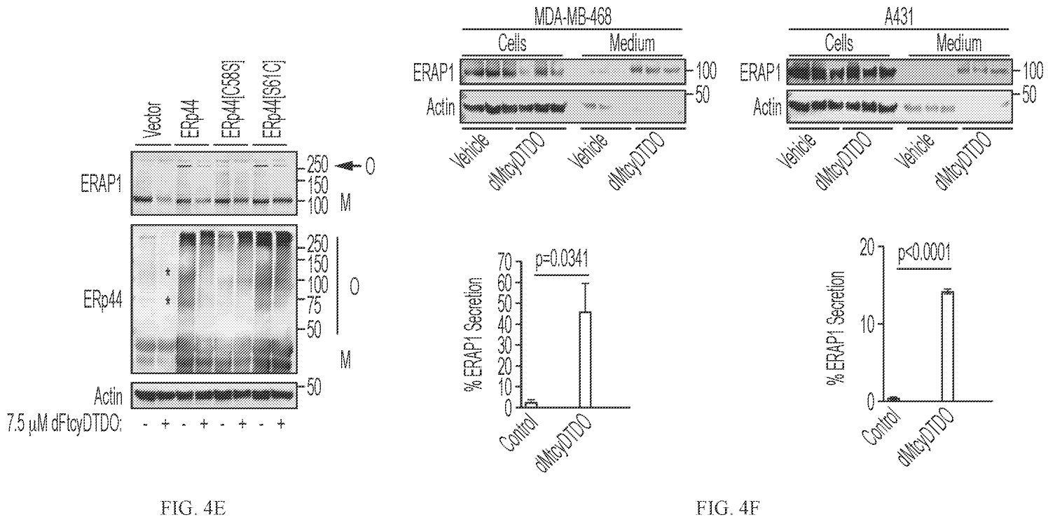

ERp44 carries out two important roles by disulfide bonding with client proteins and cycling between the Golgi and the ER as modeled in FIG. 4A. First, ERp44 retrotranslocates inappropriately oligomerized proteins from the Golgi to ER for additional rounds of disulfide bond formation and shuffling. Second, ERp44 forms disulfide bonds with select partner proteins to retain them in the ER/Golgi, preventing their secretion. To determine if DDAs block ERp44 retention of partners by binding to the ERp44 active site, MDA-MB-468 cells were treated with dMtcyDTDO for 24 h and the levels of the ERp44 partner protein ERAP1 were examined. DMtcyDTDO treatment decreased cellular ERAP1 levels (FIG. 4B). Quantitation of dMtcyDTDO effects on ERAP1 levels and dMtcyDTDO binding to its client proteins across multiple experiments showed that dMtcyDTDO decreased ERAP1 levels and ERp44 binding to client proteins in a statistically significant manner (FIG. 4C, D). ERAP1 migrates primarily as a monomer even under nonreducing conditions, indicating that ERAP1 disulfide bonding with ERp44 is transient. Enforced expression of wild type ERp44 (CXXS) resulted in the formation of a higher molecular mass band recognized by ERAP1 antibodies (FIG. 4E, arrow). This band was not observed when the C58S ERp44 active site mutant (SXXS) was expressed, but was observed when the S61C mutant (CXXC) was expressed. The ERAP1-ERp44 disulfide-bonded dimer was not observed when analyses were performed under reducing conditions (not shown) and was reduced if the cells were treated with the DDA dFtcyDTDO for 24 h (FIG. 4E).

DDA-induced secretion of ERAP1 was examined by treating MDA-MB-468 breast cancer cells or A431 skin cancer cells with dMtcyDTDO for 24 h and examining ERAP1 levels in cell extracts and in the culture medium. ERAP1 levels in the medium dramatically increased upon dMtcyDTDO treatment (FIG. 4F, upper panels), and dMtcyDTDO induced ERAP1 secretion in a statistically significant manner (FIG. 4F, lower panels).

### A Role for ERp44 in the Control of Death Receptor and EGFR Disulfide Bonding

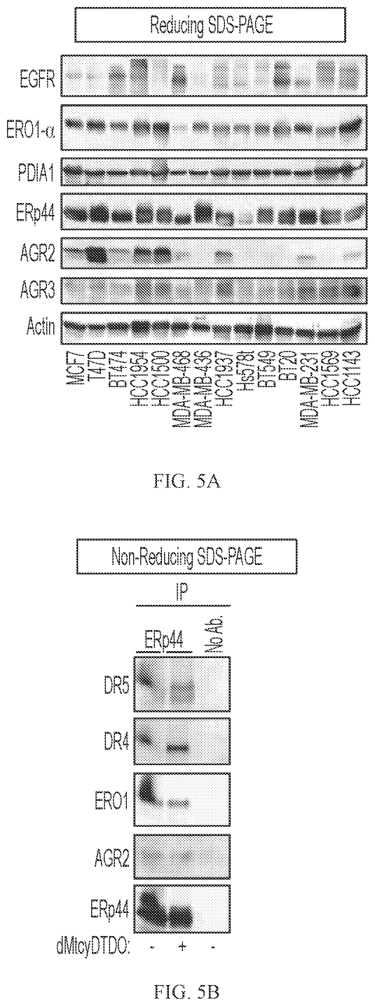

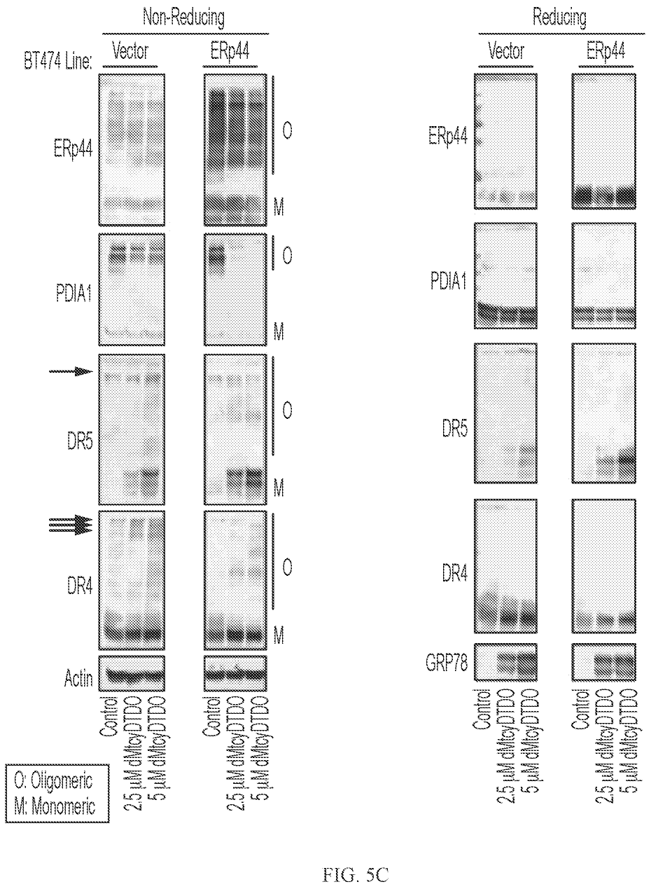

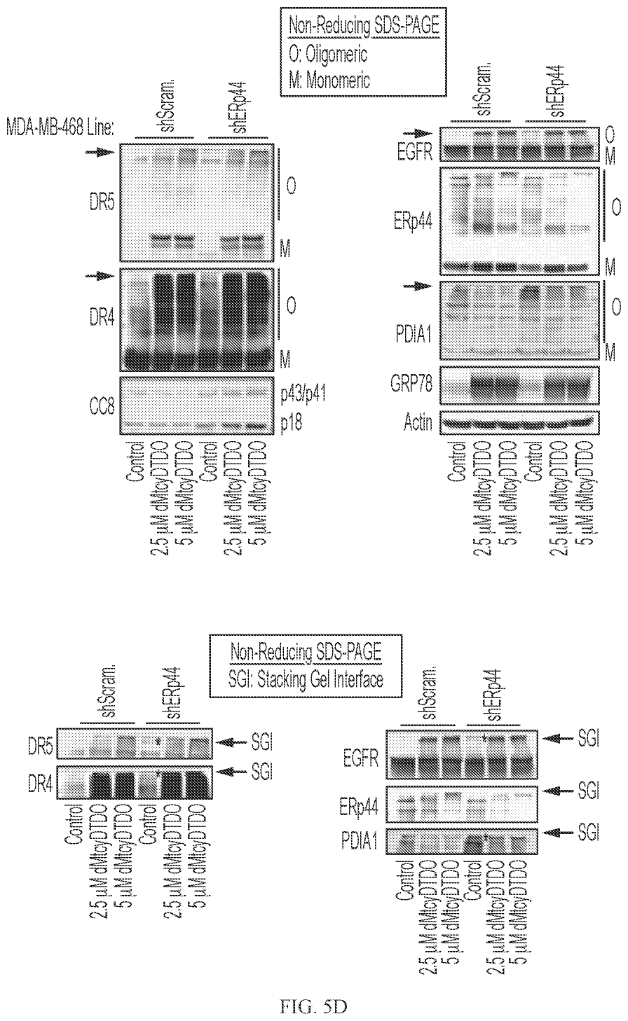

Immunoblot analysis of a panel of breast cancer cell lines revealed that PDIA1 levels were similar among the lines (FIG. 5A). In contrast, ERp44, AGR2, and AGR3 levels varied among the lines, as did the oxidoreductase ERO1, which plays a key role in producing oxidizing equivalents for disulfide bond formation. ERp44 co-immunoprecipitation experiments showed that ERp44 associates with DR5, DR4, ERO1, and AGR2 in MDA-MB-468 cells. The interactions are non-covalent because although the analyses were performed under non-reducing conditions, each protein migrated as a monomer. A BT474 cell line stably overexpres sing ERp44 was generated to examine the effect of elevated ERp44 levels on DR4/5 disulfide bonding and DDA responses. ERp44 overexpression reduced DDA induction of high molecular mass DR4 oligomers, and to a lesser extent high molecular mass DR5 oligomers (FIG. 5C, left panel, arrows). In both cases, the overall pattern was a shift from high to lower molecular mass disulfide-bonded species. These oligomeric protein forms were not observed when the analyses were performed under reducing conditions (FIG. 5C, right panel). Next, ERp44 knockdown MDA-MB-468 cells were generated and the effect on DR4/5 disulfide bonding examined. Although modest ERp44 knockdown was achieved in multiple independent experiments, ERp44 knockdown consistently increased high molecular mass oligomers of DR4, DR5, and EGFR at the interface between the stacking gel and the resolving gel in the absence of DDA treatment (FIG. 5D, left panel, arrows; expanded view in FIG. 5D, right panel, asterisks). ERp44 knockdown increased PDIA1 oligomerization and increased dMtcyDTDO induction of Caspase 8 cleavage/activation (CC8). Together, the results of the AGR2 and ERp44 knockdown experiments are consistent with roles for AGR2 and ERp44 in facilitating native disulfide bond formation of DR4 and DR5, and DDAs blocking these AGR2 and ERp44 functions.

Whether mutations within the thioredoxin-like repeat of ERp44 alter DR5 levels and disulfide bonding, and other DDA responses were examined next. Overexpressed, wild type ERp44 exhibited a high level of disulfide bond-mediated complex formation with client proteins (FIG. 6A). Both endogenous and overexpressed ERp44 monomers migrated as a doublet. This was previously shown to result from differential O-linked glycosylation. In contrast, the DR5 monomer doublet is caused by alternative splicing. The C58S ERp44 mutant (SXXS), which lacks a Cys residue in its thioredoxin-like repeat, showed lower binding to client proteins, although the levels of the monomer were similar to that of the wild type protein. The S61C ERp44 mutant (CXXC) exhibited slightly increased oligomerization compared with wild type ERp44.

DDAs were previously shown to alter the electrophoretic mobility of EGFR and CUB Domain-Containing Protein 1 (CDCP1), block the formation of complexes between these two proteins, and alter their tyrosine phosphorylation patterns. Overexpression of both ERp44 point mutants was associated with increased oligomerization of EGFR and CDCP1, but did not alter the ratio between the full length (FL) and cleaved (Cl.) forms of CDCP1. Overexpressed ERp44 exhibited disulfide bonding to client proteins (e.g., bands marked by asterisks) and these interactions were partially blocked by dFtcyDTDO treatment (FIG. 6B). As observed in FIG. 6A, the C58S mutant showed decreased binding to client proteins. The S61C ERp44 mutant bound client proteins similarly to the wild type protein, but was less sensitive to disruption of binding to its clients by dFtcyDTDO. ERp44 overexpression had little effect on DR5 levels with or without dFtcyDTDO treatment. In contrast, overexpression of either the C58S or S61C ERp44 mutants modestly increased steady-state DR5 expression and potentiated DR5 upregulation by dFtcyDTDO. Overexpression of ERp44 and its mutants modestly increased PDIA1 levels and binding to its client proteins. Consistent with the C58S ERp44 mutant inducing the strongest potentiation of DR5 upregulation by dFtcyDTDO, expression of this mutant was also associated with partial oligomerization of EGFR in vehicle treated cells that correlated with increased expression of the ERS marker GRP78 (FIG. 6B). Overall, these observations indicate that ERp44^{C58S}, and to a lesser extent, ERp44^{S61C}, interfere with disulfide bond formation of ERp44 clients, resulting in higher baseline ERS and potentiating DDA effects on DR4 and DR5. However, these observations do not explain why cancer cells that overexpress EGFR or HER2 have heightened DDA sensitivity. T47D breast cancer cells express low levels of EGFR, HER2, and HER3. Thus, T47D cells were previously used as a model system to study the effects of EGFR overexpression on DDA responses. Examination of ERp44 and PDIA1 disulfide bonding in control and EGFR overexpressing T47D cells showed that EGFR overexpression resulted in recruitment of ERp44 and PDIA1, and to a lesser extent DR4/5, into disulfide-bonded protein complexes (FIG. 6C). Under these conditions, levels of monomeric ERp44 and PDIA1 are reduced. The monomeric forms of ERp44 and PDIA1 are accessible to facilitate native disulfide bonding of their client proteins. Thus, EGFR may increase DDA sensitivity by reducing the pools of monomeric ERp44 and PDIA1 that DDAs must inactivate in order to interfere with native disulfide bond formation of ERp44 and PDIA1 client proteins.

ERp44 mediates quality control of Adiponectin and Immunoglobulin M maturation by shuttling inappropriately disulfide bonded protein forms from the Golgi back into the ER, while permitting secretion of correctly disulfide bonded proteins. It was hypothesized that ERp44 may carry out a similar function to facilitate trafficking and folding of transmembrane proteins that have disulfide bonds in their extracellular domains. Cell labeling using membrane impermeable, protein-reactive probes is useful for confirming the presence of receptors at the cell surface. Surface biotinylation studies performed on cells after treatment with dMtcyDTDO showed increased cell surface levels of DR4 and DR5, irrespective of whether labeling was performed with amine- or thiol-reactive cell-impermeable biotinylation probes (FIG. 6D). This increased accumulation of DR4/5 at the cell surface is consistent with previous findings that DDAs synergize with the DR4/5 ligand TRAIL to induce cancer cell apoptosis.

### DDAs Potentiate Non-Covalent Binding of DR5 to ERp44

Data in FIGS. 5 and 6 showing that ERp44 knockdown and overexpression of ERp44 mutants alter DR5 levels or oligomerization suggested that DR5 may be an ERp44 client protein. It was reasoned that if DR5 is an ERp44 client, the two proteins may bind to each other. ERp44 co-immunoprecipitation experiments showed that ERp44 associates with DR5 and to a lesser extent DR4 in MDA-MB-468 cells, and these interactions were potentiated by DDA treatment (FIG. 7A). Unexpectedly, these were non-covalent ERp44/DR5 complexes because although the analyses were performed under non-reducing conditions, each protein migrated as a monomer. ERp44 co-immunoprecipitation studies were next carried out in cells ectopically expressing ERp44 or the C58S or S61C ERp44 mutants. DR5 immunoprecipitated with wild type ERp44 and the S61C ERp44 mutant, but not the C58S ERp44 mutant (FIG. 7B, left panel). DDA treatment increased DR5 and decreased PDIA1 association with ERp44, which may be partly explained by changes in PDIA1 and ERp44 expression levels (FIG. 7B, right panel). Since DDAs increase DR5 levels, which could account for the increased DR5 binding to ERp44, the tet-ON system was used to drive transient high-level DR4 or DR5 expression in the absence of DDA treatment. Doxycycline-induced DR4 expression increased DR4 binding to ERp44 and binding was further enhanced by DDA treatment (FIG. 7C). Doxycycline-induced DR5 expression caused detectable DR5 binding to ERp44, which was strongly enhanced by DDA treatment. The results in FIG. 7A-C demonstrate that DR5 forms a non-covalent complex with ERp44 and that formation of this complex is strongly enhanced by DDA treatment. To determine if ERp44 in dMtcyDTDO-treated cells associates with the disulfide bond-rich extracellular domain of DR5, ERp44 immunoprecipitation experiments were performed in cells that inducibly express wild type DR5 or the Δ81-178 DR5 deletion construct lacking part of the disulfide-rich region. The results indicate that while wild type DR5 bound ERp44 in the context of dMtcyDTDO treatment, ERp44 binding to DR5[Δ81-178] was barely detectable (FIG. 7D). This result suggests that DDA binding to the ERp44 active site Cys residue not only inactivates its catalytic function, but increases its association with the disulfide-rich region of DR5. These catalytically null ERp44(DDA)-DR4/5 complexes may sequester DR4/5, preventing catalytically active forms of ERp44, PDIA1, or AGR2 from chaperoning native DR5 disulfide bond formation.

The results presented thus far indicate that DDAs alter the disulfide bonding status of DR4 and DR5, which correlates with inhibition of ERp44 and PDIA1 binding to their client proteins. However, it is unclear if genetic perturbation of DR5 disulfide bonding mimics the effects of DDAs on DR5, which include upregulation of DR5 protein levels and increased pro-apoptotic signaling. This issue was addressed by inducibly expressing DR5 mutants in which a Cys residue was mutated to Ser to disrupt one of the seven extracellular DR5 disulfide bonds. The C119S mutant was constructed to disrupt the center/fourth disulfide bond, and the C160S mutant was constructed to disrupt the seventh, most C-terminal DR5 disulfide bond. As observed previously using this doxycycline-inducible expression system, induction of wild type DR5 expression weakly increased DR5 expression (FIG. 7E). However, dMtcyDTDO/doxycycline co-treatment further increased DR5 expression, demonstrating DR5 stabilization by DDA treatment. The C119S and C160S DR5 mutants were highly expressed in both the monomeric and oligomeric forms, and this was associated with Caspase 8 cleavage/activation. DMtcyDTDO treatment modestly increased Caspase 8 cleavage, but did not further increase expression of the C119S or C160S DR5 mutants. Expression of the DR5 mutants did not upregulate the ERS marker GRP78, indicating that the mutation-driven upregulation of DR5 was not a result of ER stress induction. These results indicate that disruption of individual DR5 disulfide bonds is sufficient to promote high-level DR5 protein expression and pro-apoptotic signaling that is not further enhanced by DDA treatment.

### Certain Embodiments

1. A method of determining the ratio of oligomeric forms of one of ERp44, PDIA1, or AGR2 relative to the monomeric forms of that protein in a subject sample comprising: (a) measuring the amount of oligomeric form of one of ERp44, PDIA1, or AGR2 in the subject sample; (b) measuring the amount of monomeric form of one of ERp44, PDIA1, or AGR2 in the subject sample; (c) comparing the measurement of (a) and the measurement of (b).
2. The method of embodiment 1, wherein the measuring comprises by non-reducing immunoblot analysis of subject tumor tissue samples.
3. The method of embodiment 1, further comprising reporting the result of step (c).
4. The method of embodiment 1, wherein if the ratio of the measurements in (a)/(b) is greater than 1, then the subject is identified as in need of cancer therapy.
5. The method of embodiment 4, wherein the cancer therapy is administration of an inhibitor of ERp44, PDIA1, or AGR2.
6. A method of lowering the ratio of oligomeric forms of one of ERp44, PDIA1, or AGR2 relative to the monomeric forms of that protein in a subject comprising administering a compound to the subject, wherein the subject is determined to have a high ratio (e.g., greater than 1) of oligomeric forms of one of ERp44, PDIA1, or AGR2 relative to the monomeric forms of that protein.
7. The method of embodiment 6, wherein the compound is an inhibitor of ERp44, PDIA1, or AGR2.
8. A method of treating a patient comprising administering a cancer therapy to the patient, wherein the patient is determined to have a high ratio (e.g., greater than 1) of oligomeric forms of one of ERp44, PDIA1, or AGR2 relative to the monomeric forms of that protein.
9. The method of embodiment 8, wherein the cancer therapy is an inhibitor of ERp44, PDIA1, or AGR2.
10. The method of any of embodiments 4-9, wherein the cancer therapy is chemotherapy.
11. The method of any of embodiments 4-9, wherein the compound or cancer therapy is a DDA.
12. A test panel comprising an agent to assess the presence of ERp44 and an agent to assess the presence of PDIA1 in a subject sample.
13. The test panel of embodiment 12, further comprising an agent to assess the presence of AGR2 or AGR3.
14. The test panel of embodiment 12 or 13 further comprising an agent to assess the presence of one or more of EGFR, HER2, HER3, DR4, and DR5.
15. The test panel of any of embodiments 12-14, wherein the agent is a small molecule or an antibody.
16. The test panel of any of embodiments 12-15, wherein the agent is a detectably labelled small molecule or a detectably labelled antibody.
17. The test panel of any of embodiments 12-16, wherein the agent is a labelled DDA.
18. A method for testing for the presence of ERp44 and PDIA1 in a subject sample comprising contacting the test panel of any of embodiments 12-17 with a subject sample.
19. The method of embodiment 18, wherein the sample is tissue, cell, blood, saliva, sputum, serum, or plasma.
20. The method of any of embodiments 18-19, further comprising testing for the presence of AGR2 or AGR3 in the sample.
21. The method of any of embodiments 18-20, comprising reporting the presence or absence of ERp44 and PDIA1 in the subject sample.
22. The method of any of embodiments 18-21, comprising reporting the presence or absence of AGR2 or AGR3 in the sample.
23. The method of any of embodiments 18-22, comprising testing for the presence of one or more of EGFR, HER2, HER3, DR4, and DR5 in the sample.
24. The method of any of embodiments 18-23, comprising reporting the presence or absence of one or more of EGFR, HER2, HER3, DR4, and DR5 in the sample.
25. The method of any of embodiments 1-11, wherein the cancer is breast cancer.
26. The method of any of embodiments 1-11, wherein the subject is identified as having a cancer with likely enhanced response to therapy by administration of an inhibitor of ERp44, PDIA1, or AGR2.

What is claimed is:

1. A method of determining the ratio of oligomeric forms of one of ERp44, PDIA1, or AGR2 relative to the monomeric form of that same protein in a biological sample of a subject, the method comprising: (a) measuring the amount of oligomeric forms of one of ERp44, PDIA1, or AGR2 in the subject sample; (b) measuring the amount of monomeric form of the same protein measured in step (a) in the subject sample; and (c) comparing the measurement of (a) and the measurement of (b) to determine the ratio of oligomeric forms to monomeric form.

2. The method of claim 1, wherein the measuring comprises non-reducing immunoblot analysis of subject tumor tissue samples.

3. The method of claim 1 or 2, further comprising reporting the result of step (c).

4. The method of any of claims 1-3, wherein if the ratio determined in step (c) is greater than a ratio of measurements in (a)/(b) taken from a healthy subject sample, then the subject is identified as in need of cancer therapy.

5. The method of claim 4, wherein the cancer therapy is administration of an inhibitor of ERp44, PDIA1, or AGR2.

6. The method of any of claims 1-4, wherein if the ratio determined in step (c) is greater than or equal to 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.5, 3.0., 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 100, 200, 300, 400, 500, 1000, 10000, 100000, or 1000000, then the subject is identified as in need of cancer therapy.

7. A method of lowering the ratio of oligomeric forms of one of ERp44, PDIA1, or AGR2 relative to the monomeric form of that same protein in a biological sample of a subject, the method comprising administering a compound to the subject, wherein the subject is determined to have a high ratio of oligomeric forms of one of ERp44, PDIA1, or AGR2 relative to the monomeric form of that same protein.

8. The method of claim 7, wherein the compound is an inhibitor of ERp44, PDIA1, or AGR2.

9. The method of claim 7 or 8, wherein a high ratio of oligomeric forms to monomeric form is a ratio greater than a ratio of oligomeric forms to monomeric form of that same protein taken from a healthy subject sample.

10. The method of any of claims 7-9, wherein a high ratio of oligomeric forms to monomeric form is a ratio greater than or equal to 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.5, 3.0., 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 100, 200, 300, 400, 500, 1000, 10000, 100000, or 1000000.

11. A method of treating a patient comprising administering a cancer therapy to the patient, wherein the patient is determined to have a high ratio of oligomeric forms of one of ERp44, PDIA1, or AGR2 relative to the monomeric form of that same protein.

12. The method of claim 11, wherein the cancer therapy is an inhibitor of ERp44, PDIA1, or AGR2.

13. The method of claim 11 or 12, wherein a high ratio of oligomeric forms to monomeric form is a ratio greater than a ratio of oligomeric forms to monomeric form of that same protein taken from a healthy subject sample.

14. The method of any of claims 7-9, wherein a high ratio of oligomeric forms to monomeric form is a ratio greater than or equal to 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.5, 3.0., 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, 100, 200, 300, 400, 500, 1000, 10000, 100000, or 1000000.

15. The method of any of claims 1-14, wherein the cancer is glioblastoma, pancreatic cancer, or breast cancer.

16. The method of any of claims 1-15, wherein the cancer is breast cancer.

17. The method of any of claims 1-16, wherein the cancer is HER2-positive breast cancer.

18. The method of any of claims 1-16, wherein the cancer is triple-negative breast cancer.

19. The method of any of claims 1-18, wherein the subject is identified as having a cancer expected to have enhanced response to therapy by administration of an inhibitor of ERp44, PDIA1, or AGR2.

20. The method of any of claims 4-19, wherein the cancer therapy is chemotherapy.

21. The method of any of claims 4-19, wherein the compound or cancer therapy is a DDA.

22. The method of claim 21, wherein the DDA is of Formula (I): 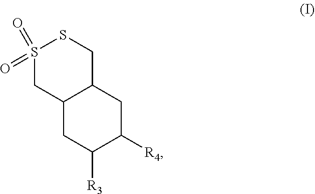
or a salt thereof, wherein:
(iv) R₃ is halo or haloalkoxy; and R₄ is halo or haloalkoxy; or
(v) R₃ is H or C₁-C₆ alkoxy; and R₄ is halo or haloalkoxy; or
(vi) R₃ is halo or haloalkoxy; and R₄ is H or C₁-C₆ alkoxy.

23. The method of claim 21, wherein the DDA is of Formula (II): 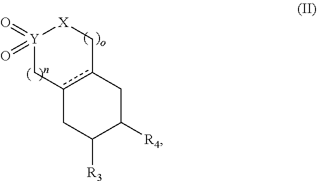
or a salt thereof, wherein:
X is S or Se;
Y is S or Se;
R₃ is selected from H or C₁-C₆ alkoxy;
R₄ is selected from H or C₁-C₆ alkoxy;
n is 0, 1, 2, or 3;
o is 0, 1, 2, or 3; and [symbol]
denotes a carbon-carbon single bond or double bond;
wherein if [symbol] is a single bond, X and Y are both S, and n and o are each 1, then at least one of R₃ or R₄ is C₁-C₆ alkoxy.

24. A test panel comprising an agent to assess the presence of ERp44 and/or an agent to assess the presence of PDIA1 in a subject sample.

25. The test panel of claim 24 comprising an agent to assess the presence of ERp44 and an agent to assess the presence of PDIA1 in a subject sample.

26. The test panel of claim 24 or 25, further comprising an agent to assess the presence of AGR2 or AGR3.

27. The test panel of any of claims 24-26 further comprising an agent to assess the presence of one or more of EGFR, HER2, HER3, DR4, and DR5.

28. The test panel of any of claims 24-27, wherein the agent is a small molecule or an antibody.

29. The test panel of any of claims 24-28, wherein the agent is a detectably labelled small molecule or a detectably labelled antibody.

30. The test panel of any of claims 24-29, wherein the agent is a detectably labelled DDA.

31. The test panel of claim 30, wherein the detectably labelled DDA is of Formula (III): 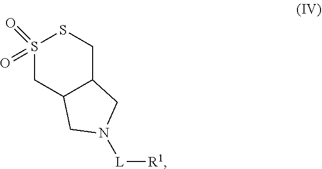
or a salt thereof, wherein:
L is a bond, substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, or —OC(═O)N(R^{A})—, or a combination thereof;
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or two R^{A} groups are joined to form a substituted or unsubstituted heterocyclic ring; and
R¹ is hydrogen, a nitrogen protecting group, or a label.

32. The test panel of claim 31, wherein R¹ is a label.

33. The test panel of claim 31, wherein R¹ is biotin.

34. A method of testing for the presence of ERp44 and/or PDIA1 in a subject sample comprising contacting the test panel of claim 24 with a subject sample.

35. The method of claim 34 comprising testing for the presence of ERp44 and PDIA1 in a subject sample comprising contacting the test panel of claim 24 with a subject sample.

36. The method of claim 34 or 35 comprising testing for the presence of oligomeric and monomeric forms of ERp44 and/or PDIA1 in a subject sample comprising contacting the test panel of claim 24 with a subject sample.

37. The method of any of claims 34-36, wherein the sample is tissue, cell, blood, saliva, sputum, serum, or plasma.

38. The method of any of claims 34-37 further comprising testing for the presence of AGR2 or AGR3 in the sample.

39. The method of any of claims 34-38, comprising reporting the presence or absence of ERp44 and/or PDIA1 in the subject sample.

40. The method of any of claims 34-39, comprising reporting the presence or absence of oligomeric and monomeric forms of ERp44 and/or PDIA1 in the subject sample.

41. The method of any of claims 38-40, comprising reporting the presence or absence of AGR2 or AGR3 in the sample.

42. The method of any of claims 34-41, comprising testing for the presence of one or more of EGFR, HER2, HER3, DR4, and DR5 in the sample.

43. The method of any of claims 34-42, comprising reporting the presence or absence of one or more of EGFR, HER2, HER3, DR4, and DR5 in the sample.

44. A compound of Formula (III): 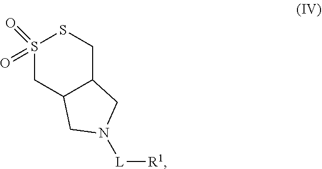
or a salt thereof, wherein:
L is a bond, substituted or unsubstituted alkylene, substituted or unsubstituted alkenylene, substituted or unsubstituted alkynylene, substituted or unsubstituted carbocyclylene, substituted or unsubstituted heterocyclylene, substituted or unsubstituted arylene, substituted or unsubstituted heteroarylene, substituted or unsubstituted heteroalkylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(═O)—, —OC(═O)O—, or —OC(═O)N(R^{A})—, or a combination thereof;
each occurrence of R^{A} is, independently, hydrogen, substituted or unsubstituted acyl, substituted or unsubstituted alkyl, substituted or unsubstituted alkenyl, substituted or unsubstituted alkynyl, substituted or unsubstituted heteroalkyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, or two R^{A} groups are joined to form a substituted or unsubstituted heterocyclic ring; and
R¹ is hydrogen, a nitrogen protecting group, or a label.


SEQUENCE LISTING (19 sequences)

SEQ ID NO: 1 - DNA, 33 bp, Artificial Sequence
  Synthetic
ttttggatcccaccatgcatcctgccgtcttcc

SEQ ID NO: 2 - DNA, 32 bp, Artificial Sequence
  Synthetic
ttttctcgagttaaagctcatctcgatccctc

SEQ ID NO: 3 - DNA, 47 bp, Artificial Sequence
  Synthetic
ctttagtaaatttttatgctgactggagtcgtttcagtcagatgttg

SEQ ID NO: 4 - DNA, 47 bp, Artificial Sequence
  Synthetic
caacatctgactgaaacgactccagtcagcataaaaatttactaaag

SEQ ID NO: 5 - DNA, 40 bp, Artificial Sequence
  Synthetic
gactggtgtcgtttctgtcagatgttgcatccaatttttg

SEQ ID NO: 6 - DNA, 40 bp, Artificial Sequence
  Synthetic
caaaaattggatgcaacatctgacagaaacgacaccagtc

SEQ ID NO: 7 - DNA, 32 bp, Artificial Sequence
  Synthetic
ttttggatcccaccatggagaaaattccagtg

SEQ ID NO: 8 - DNA, 28 bp, Artificial Sequence
  Synthetic
tttttacgtattacaattcagtcttcag

SEQ ID NO: 9 - DNA, 30 bp, Artificial Sequence
  Synthetic
ttttggatcccagagataccacagtcaaac

SEQ ID NO: 10 - DNA, 26 bp, Artificial Sequence
  Synthetic
ttttgtcgacttacaattcagtcttc

SEQ ID NO: 11 - DNA, 32 bp, Artificial Sequence
  Synthetic
catcacttggatgagtccccacacagtcaagc

SEQ ID NO: 12 - DNA, 32 bp, Artificial Sequence
  Synthetic
gcttgactgtgtggggactcatccaagtgatg

SEQ ID NO: 13 - protein, 4 aa, Artificial Sequence
  Synthetic
  misc_feature (2)..(3): Xaa can be any naturally occurring amino acid
SXXS

SEQ ID NO: 14 - protein, 4 aa, Artificial Sequence
  Synthetic
  misc_feature (2)..(3): Xaa can be any naturally occurring amino acid
CXXC

SEQ ID NO: 15 - protein, 4 aa, Artificial Sequence
  Synthetic
CPHS

SEQ ID NO: 16 - protein, 4 aa, Artificial Sequence
  Synthetic
CQYS

SEQ ID NO: 17 - protein, 4 aa, Artificial Sequence
  Synthetic
CRFS

SEQ ID NO: 18 - protein, 4 aa, Artificial Sequence
  Synthetic
  misc_feature (2)..(3): Xaa can be any naturally occurring amino acid
CXXS

SEQ ID NO: 19 - protein, 5 aa, Artificial Sequence
  Synthetic
  misc_feature (3)..(4): Xaa can be any naturally occurring amino acid
SCXXS